SEQUENCE LISTING (27 sequences)

SEQ ID NO: 1 - DNA, 1470 bp, Homo sapiens
atggctctcctctttctcctaccccttgtcatgcagggtgtgagcagggctgagatgggcaccgcggatctggggccgtcctcagtgcctacaccaactaatgttacaattgaatcctataacatgaaccctatcgtatattgggagtaccagatcatgccacaggtccctgtttttaccgtagaggtaaagaactatggtgttaagaattcagaatggattgatgcctgcatcaatatttctcatcattattgtaatatttctgatcatgttggtgatccatcaaattctctttgggtcagagttaaagccagggttggacaaaaagaatctgcctatgcaaagtcagaagaatttgctgtatgccgagatggaaaaattggaccacctaaactggatatcagaaaggaggagaagcaaatcatgattgacatatttcacccttcagtttttgtaaatggagacgagcaggaagtcgattatgatcccgaaactacctgttacattagggtgtacaatgtgtatgtgagaatgaacggaagtgagatccagtataaaatactcacgcagaaggaagatgattgtgacgagattcagtgccagttagcgattccagtatcctcactgaattctcagtactgtgtttcagcagaaggagtcttacatgtgtggggtgttacaactgaaaagtcaaaagaagtttgtattaccattttcaatagcagtataaaaggttctctttggattccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatga

SEQ ID NO: 2 - protein, 489 aa, Homo sapiens
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 3 - DNA, 1014 bp, Homo sapiens
atgcgaccgacgctgctgtggtcgctgctgctgctgctcggagtcttcgccgccgccgccgcggccccgccagaccctctttcccagctgcccgctcctcagcacccgaagattcgcctgtacaacgcagagcaggtcctgagttgggagccagtggccctgagcaatagcacgaggcctgttgtctaccaagtgcagtttaaatacaccgacagtaaatggttcacggccgacatcatgtccataggggtgaattgtacacagatcacagcaacagagtgtgacttcactgccgccagtccctcagcaggcttcccaatggatttcaatgtcactctacgccttcgagctgagctgggagcactccattctgcctgggtgacaatgccttggtttcaacactatcggaatgtgactgtcgggcctccagaaaacattgaggtgaccccaggagaaggctccctcatcatcaggttctcctctccctttgacatcgctgatacctccacggcctttttttgttattatgtccattactgggaaaaaggaggaatccaacaggtcaaaggccctttcagaagcaactccatttcattggataacttaaaaccctccagagtgtactgtttacaagtccaggcacaactgctttggaacaaaagtaacatctttagagtcgggcatttaagcaacatatcttgctacgaaacaatggcagatgcctccactgagcttcagcaagtcatcctgatctccgtgggaacattttcgttgctgtcggtgctggcaggagcctgtttcttcctggtcctgaaatatagaggcctgattaaatactggtttcacactccaccaagcatcccattacagatagaagagtatttaaaagacccaactcagcccatcttagaggccttggacaaggacagctcaccaaaggatgacgtctgggactctgtgtccattatctcgtttccggaaaaggagcaagaagatgttctccaaacgctttga

SEQ ID NO: 4 - protein, 337 aa, Homo sapiens
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 5 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 6 - DNA, 501 bp, Homo sapiens
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 7 - RNA, 32 nt, Artificial sequence
  PCR sense primer for generation of aptamers
gggaggacgaugcggncagacgacucgcccga

SEQ ID NO: 8 - DNA, 32 bp, Artificial sequence
  SELEX PCR sense primer
taatacgactcactatagggaggacgatgcgg

SEQ ID NO: 9 - DNA, 16 bp, Artificial sequence
  SELEX PCR sense primer
tcgggcgagtcgtctg

SEQ ID NO: 10 - DNA, 26 bp, Artificial sequence
  IFN gamma aptamer
ggggttggttgtgttgggtgttgtgt

SEQ ID NO: 11 - DNA, 39 bp, Artificial sequence
  IFN gamma aptamer
cagguaauuacaugaagguggguuagguacuuucagggu

SEQ ID NO: 12 - DNA, 903 bp, Homo sapiens
atgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactga

SEQ ID NO: 13 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 14 - DNA, 978 bp, Artificial sequence
  nucleotide sequence encoding mouse IFN gamma R1-cartilage oligomeric matrix protein chimera
atgggcccgcaggcggcagctggcaggatgattctgctggtggtcctgatgctgtctgcgaaggtcgggagtggagctttgacgagcaccgaggatcctgagcctccctcggtgcctgtaccgacgaatgttctaattaagtcttataacttgaaccctgtcgtatgctgggaataccagaacatgtcacagactcctatttttactgtacaggtaaaggtgtattcgggttcctggactgattcctgcaccaacatttctgatcattgttgtaatatctatggacaaattatgtatcctgatgtatctgcctgggccagagttaaagctaaggttggacaaaaagaatctgactatgcacggtcaaaagagttccttatgtgcctaaagggaaaggtcgggccccctggcctggagatcaggaggaagaaggaagaacagctctccgtcctcgtatttcaccctgaagtcgttgtgaatggagagagccagggaaccatgtttggtgacgggagcacctgttacacattcgactatactgtgtatgtggagcataaccggagtggggagatcctacatacgaaacatacggtcgaaaaagaagagtgtaatgagactctgtgtgagttaaacatctcagtatccacactggattccagatattgtatttcagtagacggaatctcatctttctggcaagttagaacagaaaaatcgaaagacgtctgtatccctcctttccatgatgacagaaaggagcggccgcagcctcagcctcagcctaagcctcagcctaagcctgagcctgagggagacctagccccacagatgcttcgagaactccaggagactaatgcggcgctgcaagacgtgagagagctcttgcgacacagggtcaaggagatcaccttcctgaagaatacggtgatggaatgtgacgcttgcggatacccatacgacgtaccagattacgcttag

SEQ ID NO: 15 - protein, 325 aa, Artificial sequence
  amino acid sequence of mouse IFN gamma R1-cartilage oligomeric matrix protein chimera
MGPQAAAGRMILLVVLMLSAKVGSGALTSTEDPEPPSVPVPTNVLIKSYNLNPVVCWEYQNMSQTPIFTVQVKVYSGSWTDSCTNISDHCCNIYGQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCISVDGISSFWQVRTEKSKDVCIPPFHDDRKERPQPQPQPKPQPKPEPEGDLAPQMLRELQETNAALQDVRELLRHRVKEITFLKNTVMECDACGYPYDVPDYA

SEQ ID NO: 16 - DNA, 1107 bp, Artificial sequence
  Sequence encoding for DcR3-COMP
atgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcaccctcagcctcagcctaagcctcagcctaagcctgagcctgagggagacctagccccacagatgcttcgagaactccaggagactaatgcggcgctgcaagacgtgagagagctcttgcgacacagggtcaaggagatcaccttcctgaagaatacggtgatggaatgtgacgcttgcggatacccatacgacgtaccagattacgct

SEQ ID NO: 17 - protein, 369 aa, Artificial sequence
  Sequence for chimeric protein DcR3-COMP
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVHPQPQPKPQPKPEPEGDLAPQMLRELQETNAALQDVRELLRHRVKEITFLKNTVMECDACGYPYDVPDYA

SEQ ID NO: 18 - DNA, 207 bp, Artificial sequence
  nucleotide sequence encoding a coiled-coil domain of cartilage oligomeric matrix protein
cctcagcctcagcctaagcctcagcctaagcctgagcctgagggagacctagccccacagatgcttcgagaactccaggagactaatgcggcgctgcaagacgtgagagagctcttgcgacacagggtcaaggagatcaccttcctgaagaatacggtgatggaatgtgacgcttgcggatacccatacgacgtaccagattacgct

SEQ ID NO: 19 - protein, 69 aa, Artificial sequence
  amino acid sequence of a coiled-coil domain of cartilage oligomeric matrix protein
PQPQPKPQPKPEPEGDLAPQMLRELQETNAALQDVRELLRHRVKEITFLKNTVMECDACGYPYDVPDYA

SEQ ID NO: 20 - DNA, 610 bp, Artificial sequence
  Phosphoglycerokinase 1 promoter
cgcgcgatcacgagactagcctcgacgatggtcgagtaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctctagcgggatcggattcgaatcccggccgggaacggtgcattggaacgcggattccccgtgccaag

SEQ ID NO: 21 - DNA, 640 bp, Homo sapiens
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagcc

SEQ ID NO: 22 - DNA, 672 bp, Artificial sequence
  chimeric CMV-chicken beta-actin promoter
gtgccacctggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 23 - DNA, 776 bp, Artificial sequence
  Cytomegalovirus promoter
gtagccatgctctggaagatcttcaatatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactaga

SEQ ID NO: 24 - protein, 14 aa, Artificial sequence
  amino acid sequence of linker peptide comprised in the sequence of the coiled-coil domain of COMP
PQPQPKPQPKPEPE

SEQ ID NO: 25 - protein, 9 aa, Artificial sequence
  Amino acid sequence of Hemagglutinin tag sequence comprised in sequence of the coiled-coil domain of COMP
YPYDVPDYA

SEQ ID NO: 26 - DNA, 942 bp, Artificial sequence
  nucleotide sequence encoding human IFNgR1-COMP chimera
atggctctcctctttctcctaccccttgtcatgcagggtgtgagcagggctgagatgggcaccgcggatctggggccgtcctcagtgcctacaccaactaatgttacaattgaatcctataacatgaaccctatcgtatattgggagtaccagatcatgccacaggtccctgtttttaccgtagaggtaaagaactatggtgttaagaattcagaatggattgatgcctgcatcaatatttctcatcattattgtaatatttctgatcatgttggtgatccatcaaattctctttgggtcagagttaaagccagggttggacaaaaagaatctgcctatgcaaagtcagaagaatttgctgtatgccgagatggaaaaattggaccacctaaactggatatcagaaaggaggagaagcaaatcatgattgacatatttcacccttcagtttttgtaaatggagacgagcaggaagtcgattatgatcccgaaactacctgttacattagggtgtacaatgtgtatgtgagaatgaacggaagtgagatccagtataaaatactcacgcagaaggaagatgattgtgacgagattcagtgccagttagcgattccagtatcctcactgaattctcagtactgtgtttcagcagaaggagtcttacatgtgtggggtgttacaactgaaaagtcaaaagaagtttgtattaccattttcaatagcagtataaaaggtcctcagcctcagcctaagcctcagcctaagcctgagcctgagggagacctagccccacagatgcttcgagaactccaggagactaatgcggcgctgcaagacgtgagagagctcttgcgacacagggtcaaggagatcaccttcctgaagaatacggtgatggaatgtgacgcttgcggatacccatacgacgtaccagattacgct

SEQ ID NO: 27 - protein, 314 aa, Artificial sequence
  amino acid sequence of human IFNgR1-COMP chimera
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGPQPQPKPQPKPEPEGDLAPQMLRELQETNAALQDVRELLRHRVKEITFLKNTVMECDACGYPYDVPDYA

(19) United States
(12) Patent Application Publication
(10) Pub. No.: US 2012/0276114 A1
(43) Pub. Date: Nov. 1, 2012

(54) IFN-GAMMA INHIBITORS IN THE TREATMENT OF MOTONEURON DISEASES

(75) Inventors: Samareh Azeredo Da Silveira Lajaunias, Lausanne (CH); Julianne Aebischer, Morat (CH); Cedric Raoul, Marseille (FR)

(73) Assignees: UNIVERSITE D'AIX-MARSEILLE, Marseille Cedex 07 (FR); LASCCO SA, Geneva (CH)

(21) Appl. No.: 13/510,323

(22) PCT Filed: Nov. 18, 2010
(86) PCT No.: PCT/IB10/55253

§ 371 Date: May 17, 2012

(30) Foreign Application Priority Data
Nov. 18, 2009 (EP) 09290871.4

(51) Int. Cl. A61K 48/00; A61K 31/7088; A61K 38/21; A61P 25/00; G01N 33/566; C12Q 1/68; C40B 30/04; C12N 15/86; C12N 15/63; A61K 39/395

(52) U.S. Cl. 424/158.1; 435/320.1; 514/44 R; 514/17.7; 424/93.2; 435/7.92; 435/6.12; 435/7.1; 506/9; 435/456

(57) ABSTRACT

The present invention is directed to new compositions uses thereof and related methods for the treatment of a motoneuron disease or disorder. In particular, the invention relates to the new use of IFNγ antagonists or viral vectors, uses, compositions thereof and related methods for the treatment of motoneuron disease or disorder such as ALS.

### FIELD OF THE INVENTION

The present invention relates to the treatment of motoneuron diseases, in particular, amyotrophic lateral sclerosis (ALS).

### BACKGROUND OF THE INVENTION

ALS is an incurable adult-onset neurodegenerative disease that affects primarily upper and lower motoneurons in the brain and spinal cord. Dominant mutation in superoxide dismutase-1 (Sod1) gene is the most prominent cause of inherited ALS: approximately 10% of ALS cases have a familial history of the disease and among these, 20% are caused by a dominantly inherited mutation in the superoxide dismutase-1 (SOD1) gene. Accumulating evidence suggests that mutant SOD1-mediated damage in glial cells contributes to ALS pathogenesis by releasing factors selectively toxic to motoneurons, but the mechanistic basis of this motoneuron-specific elimination is poorly understood, impeding therefore the development of effective therapies.

Mice expressing human SOD1 mutations develop a motor syndrome with features of the human disease (Bruijn et al., 2004, Als. Annu. Rev. Neurosci., 27, 723-749). Both cell-autonomous and non-cell-autonomous processes mediated by mutant SOD1 contribute to motoneuron degeneration (Boillee et al., 2006, Neuron, 52, 39-59): a toxic action of mutant SOD1 within motoneurons has been documented as crucial for the onset and the early phase of disease progression (Boillee et al., 2006, Science, 312, 1389-1392), whereas a non-cell-autonomous component, which involves damage to astrocytes and microglia is determinant for disease progression (Yamanaka et al., 2008, Nat. Neurosci., 11, 251-253).

LIGHT (TNFSF14) is a type II transmembrane protein of the TNFR superfamily that can engage the lymphotoxin β receptor (LT-βR), the herpes virus entry mediator (HVEM) and the decoy receptor 3 (DcR3). LIGHT, which is expressed by immature dendrocytes, activated lymphocytes, monocytes, and natural killer cells, is important for both innate and adaptive immune processes. LIGHT signalling through LT-βR or HVEM serves as a co-stimulatory signal for T cell proliferation and induces secretion of various cytokines and expression of adhesion molecules. Remarkably, LIGHT can act with the immunomodulatory cytokine interferon-gamma (IFNγ) to induce a singular slow apoptotic death in tumor cells (Chen et al., 2000, J. Biol. Chem., 275, 38794-38801), which is reminiscent of the progressive nature of motoneuron degeneration in ALS.

IFNγ is an immunomodulatory cytokine produced by T lymphocytes and natural killer cells. In the central nervous system, IFNγ which is increased in chronic inflammatory disease (e.g. multiple sclerosis) or following injury, can activate astrocytes and microglial cells. Interestingly, levels of IFNγ have been shown to increase both in SOD1 mutant mice and sporadic patients (Hensley et al., 2003, Neurobiol. Dis., 14, 74-80).

The main challenging issue regarding therapeutic approaches for motoneuron diseases concerns the delivery of therapeutic message to the broadest number of motoneurons. Currently, no therapy exists for ALS. A treatment developed to reduce damage to motoneurons by decreasing the release of glutamate proved to prolong survival of ALS patients by several months, mainly in those with difficulty swallowing and to extend the time before patients need a ventilation support. However, no treatment is able to reverse the damage already done to motoneurons. Therefore, methods and compounds useful to efficiently arresting or slowing the development of motoneuron diseases and their symptoms would be particularly desirable.

### SUMMARY OF THE INVENTION

The present invention is directed towards to the new use of IFNγ antagonists in the treatment of motoneuron disease such as ALS, new compositions, and uses thereof and related methods for the treatment of ALS. In particular, the invention relates to the new use of IFNγ antagonists, such as antibodies, aptamers, chimeric proteins, or viral vectors, new compositions, and uses thereof and related methods for the treatment of motoneuron disease, such as ALS.

A first aspect of the invention provides a use of an IFNγ antagonist for the manufacture of a medicament for the treatment of a motoneuron disease or disorder.

A second aspect of the invention provides a method of treating a motoneuron disease or disorder in a subject in need thereof, comprising administering in said subject a pharmaceutical composition which comprises an IFNγ antagonist.

A third aspect of the invention provides a method for delivering a nucleic acid sequence encoding an IFNγ antagonist to cells selected from neural, microglial and meningeal cells comprising:

(a) Providing a virion comprising a viral vector, said vector comprising at least one expression control element operably linked to a nucleic acid sequence encoding for an IFNγ antagonist; (b) Bringing the virion into contact with the said cells, whereby transduction of the viral vector results in the expression of said nucleic acid sequence in the transduced cells and the expression of said nucleic acid sequence by said cells.

A fourth aspect of the invention provides a method of treating a motoneuron disease or disorder in a subject in need thereof, comprising administering in said subject a pharmaceutical composition which comprises (a) a pharmaceutically acceptable excipient; and (b) virions comprising a viral vector, said viral vector comprising a nucleic acid sequence encoding for an IFNγ antagonist, operably linked to at least one expression control element that controls expression of the said nucleic acid sequence.

A fifth aspect of the invention provides a method of treating a motoneuron disease or disorder in a subject in need thereof, comprising implanting and/or transplanting genetically engineered stem cells secreting an IFNγ antagonist in the central nervous system of said subject.

A sixth aspect of the invention provides an in vitro method for detection and/or prognosis of a motoneuron disease in a sample from a subject, comprising the following steps: (a) measuring IFNγ levels in a sample from said subject; and (b) comparing IFNγ level data obtained in step (a) to IFNγ level data of subjects suffering from a motoneuron disease wherein IFNγ levels correlate with a motoneuron disease status in said subject.

A seventh aspect of the invention provides a kit for in vitro detecting a motoneuron disease in a subject comprising: (a) at least one sample testing device that provides a readable signal proportional to IFNγ concentration in a sample; (b) an electronic monitor having reading means to read the readable signal obtained under step (a) and incorporating computer means to interpret the readable signals and to determine therefrom in conjunction with data from previous sample tests the motoneuron degenerescence status of said subject.

An eighth aspect of the invention provides a viral vector according to the invention.

A ninth aspect of the invention provides a viral vector according to the invention for use as a medicament.

A tenth aspect of the invention provides an IFNγ antagonist according to the invention for the treatment of a neuronal disease such as an ALS disorder.

An eleventh aspect of the invention provides a pharmaceutical preparation comprising at least one viral vector according to the invention and pharmaceutically acceptable carrier or excipient.

### DESCRIPTION OF THE FIGURES

FIG. 1 represents the soluble mouse recombinant IFNγ-induced motoneuron death in a dose-dependent manner, as measured by the percentage of surviving motoneurons at 48 h later as described in Example 1. A: Motoneurons cultured for 24 h and incubated with increasing concentrations of soluble mouse recombinant IFNγ from two different sources; B: IFNγ-induced death of motoneurons as mediated by LIGHT. Motoneuron survival was determined 48 h following treatment or not with LT-βR-Fc (100 ng/ml), Fas-Fc (1 μg/ml) or TNFR1-Fc (100 ng/ml) in combination or not with 250 ng/ml of IFNγ. The number of surviving motoneurons is expressed as a percentage of the number of motoneurons in the control condition (none).

FIG. 2 represents the percentage of surviving wildtype motoneurons plated on astrocyte monolayer of indicated genotype (wildtype, SOD1^{G93A}) and incubated or not with function-blocking anti-IFNγ antibodies (500 ng/ml) or LT-βR-Fc (100 ng/ml) for 48 h and expressed as the percentage of the number of motoneurons surviving on wildtype astrocyte monolayer in the absence of any treatment as described in Example 1. (ANOVA with Tukey-Kramer's post hoc test, n=3, **P<0.01, ***P<0.001). Mean values of three independent experiments performed in triplicate.

FIG. 3 represents the levels of IFNγ in sera (A) and dissociated lumbar spinal cords (B) of 13-week-old SOD1^{G93A}, SOD1^{WT} and wild-type mice, determined by an enzyme-linked immunoabsorbent (ELISA) analysis, as described in Example 2. One-way ANOVA with Tukey-Kramer's post hoc test; n=3, ***P<0.001, **P<0.01, values are presented as mean±S.D.

FIG. 4 evidences the role of IFNγ at onset and symptomatic stages of motoneuron disease in ALS mice. Levels of IFNγ increase at those stages of motoneuron disease where total protein extracts from lumbar spinal cords of wildtype and SOD1^{G93A} mice at indicated age are resolved by SDS-PAGE and probed with antibodies to IFNγ and actin as described in Example 2. IFNγ signals were quantified, normalized to actin signals and expressed as the ratio of SOD1^{G93A} to wild-type values (n=3, *P<0.05, values are means±S.D).

FIG. 5 evidences the potential of neutralizing anti-IFNγ antibodies to protect cultured motoneurons from IFNγ-induced death. Mouse motoneurons were cultured for 24 h and treated or not with recombinant mouse IFNγ (250 ng/ml) alone or in combination with indicated concentrations of anti-IFNγ antibody or with irrelevant rat IgG (0.5 μg/ml) or anti-IFNγ antibodies (0.5 μg/ml) as controls. Motoneuron survival was determined 48 h later and expressed relative to non-treated cells. Values are means±S.D of triplicates. 1: no treatment; 2: IFNγ alone; 3: anti-IFNγ antibody 0.01 μg/ml+IFNγ; 1: no treatment; 2: IFNγ alone; 3: anti-IFNγ antibody 0.01 μg/ml+IFNγ; 4: anti-IFNγ antibody 0.05 μg/ml+IFNγ; 5: anti-IFNγ antibody 0.1 μg/ml+IFNγ; 6: anti-IFNγ antibody 0.5 μg/ml+IFNγ; 7: anti-IFNγ antibody alone; 8: IgG control; 8: IgG control+IFNγ.

FIG. 6 represents the chimeric proteins constructs for recombinant adeno-associated virus (rAAVs) for astrocytic delivery of receptors and controls as described in Example 4. Schematic representation of the constructs: Extracellular part of IFNγR1, DcR3, or Fas were fused to COMP and tagged with Human influenza haemagglutin (HA) tag. COMP control was generated by deleting >80% of the extra-cellular part of Fas. Constructs are then cloned into AAV shuttle vector incorporating the control of the astrocyte specific gfaABC₁D promoter, β-globin intron, a multiple cloning site (MCS) and the human growth hormone poly-adenenylation sequence (hGH). ITR, inverted terminal repeats.

FIG. 7 evidences the efficiency of recombinant proteins IFNγR1-COMP, DcR3-COMP, and Fas-COMP (in the form of AAV viral vectors at 1.5×10⁵ TU/ml), to interfere with death induced by their respective ligand(s) or cytokine (sFasL (50 ng/ml), sLight (50 ng/ml), IFNγ (250 ng/ml)), in contrast to the negative control (COMP). Cell survival was determined 48 h later as described in Example 4. All values are expressed as the means±S.D of three independent experiments.

FIG. 8 represents the functional involvement of the IFNγ-LIGHT-LT-βR pathway in ALS pathogenesis and the behavioural and survival rescue when LIGHT is genetically deleted in SOD1^{G93A} mice. The progressive motor deficit of SOD1^{G93A}/LIGHT+/+, SOD1^{G93A}/LIGHT−/−, LIGHT+/+ and LIGHT−/− was determined by evaluating weekly the swimming performance of mice. Values are means±S.E.M.

FIG. 9 represents the sequences described in the detailed description.

### DETAILED DESCRIPTION OF THE INVENTION

The term “motoneuron disease” includes amyotrophic lateral sclerosis, Charcot's disease, Lou Gehrig's disease, other motoneuron disorders characterized by lower or upper signs of motoneuron degeneration, such as spinal muscular atrophy (SMA), Kennedy's disease (or spinobulbar muscular atrophy), hereditary spastic paraplegia, Primary lateral sclerosis, progressive muscular atrophy. ALS is a rapidly progressive, invariably fatal neurodegenerative disease characterized by the gradual degeneration and death of motoneurons. In ALS, both the upper motoneurons and the lower motoneurons degenerate or die, ceasing to send messages to muscles leading to gradual muscle weakness, muscle atrophy and muscle fasciculations. Most people with ALS die from respiratory failure, usually within 3 to 5 years from the onset of the symptoms of the disease. Further, patients may suffer from alterations in cognitive functions, decision-making and memory.

The term “effective amount” as used herein refers to an amount of at least one polypeptide or a pharmaceutical formulation thereof according to the invention that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of an IFNγ antagonist sufficient to reduce the progression of the disease, notably to reduce or inhibit the motoneuron damage process and thereby elicit the response being sought (i.e. an “inhibition effective amount”).

The term “efficacy” of a treatment according to the invention can be measured based on changes in the course of disease in response to a use or a method according to the invention. For example, the efficacy of a treatment according to the invention can be measured by a reduction of motor deficit and/or by a protective effect against motoneuron damage and the like associated with ALS. The efficacy of a treatment according to the invention can be measured by an amelioration of behavioural symtoms which should also exert a positive influence on the main symtoms typically observed in ALS patients.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, i.e., arresting its development; or relieving the disease, i.e., causing regression of the disease and/or its symptoms or conditions such as improvement or remediation of damage. In particular, the methods, uses, polypeptides and compositions according to the invention are useful in the preservation and/or restoration of at least one functional parameter selected from neuron functional integrity, motor and cognitive capacity in ALS patients.

The term “subject” as used herein refers to mammals. For examples, mammals contemplated by the present invention include human, primates, domesticated animals such as cattle, sheep, pigs, horses, laboratory rodents and the like. In a particular embodiment, the subject is a patient suffering from or susceptible to suffer from a motoneuron disease. In another particular embodiment, the subject is an animal model of a motoneuron disease.

The term “isolated” is used to indicate that the molecule is free of association with other proteins or polypeptides, for example as a purification product of recombinant host cell culture or as a purified extract.

The term “antibody” comprises antibodies, chimeric antibodies, fully human, humanized, genetically engineered or bispecific or multispecific antibodies as well as fragments thereof such as single chain antibodies (scFv) or domain antibodies, binding to IFNγ or IFNγ receptors such as IFNγ receptor 1 and/or IFNγ receptor 2 or IFNγ effectors LIGHT, LT-βR, HVEM, or DcR3, or fragments thereof and the like. Antibodies of this invention may be monoclonal or polyclonal antibodies, or fragments or derivative thereof having substantially the same antigen specificity. The term “selectively” indicates that the antibodies preferentially recognize and/or bind the target polypeptide or epitope, i.e., with a higher affinity than any binding to any other antigen or epitope, i.e. the binding to the target polypeptide can be discriminated from non-specific binding to other antigens. The binding affinity of an antibody can be readily determined by one of ordinary skill in the art, for example, by Scatchard analysis (Scatchard et al., 1949, Ann NY Acad. ScL, 51, 660-672). According to one embodiment, an anti-IFNγ antibody according to the invention is selected from R4-6A2 and 15027.

Antibodies according to the invention can be generated by immunization of a suitable host (e.g., mice, humanized mice, rats, rabbits, goats, sheeps, donkeys, monkeys). Antibodies according to the invention can also be generated using antibody display, in particular phage display, or using human B cell isolation and a transplantation of selected human B cells into immunodeficient mice. The determination of immunoreactivity with an immunogenic IFNγ polypeptide may be made by any of several methods well known in the art, including, e.g., immunoblot assay and ELISA. Modification of antibodies according to the invention into therapeutically useful derivatives may be made by methods as described in Holliger et al., 2005, Nat. Biotech., 23, 1126-1136.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

The term “inhibitor” or “antagonist” is defined as a molecule that antagonizes or inhibits completely or partially the activity of biological molecule.

The nucleotide sequence encoding human IFNγR1 is presented in SEQ ID NO: 1 (Genbank accession number NM_{—}00416) and the amino acid sequence of human IFNγR1 is described in SEQ ID NO: 2. The nucleotide sequence encoding human IFNγR2 is presented in SEQ ID NO: 3 (Genbank accession number NM_{—}005534) and the amino acid sequence of human IFNγR2 is described in SEQ ID NO: 4. As used herein, the term IFNγ encompasses human IFNγ polypeptides having an amino acid sequence of SEQ ID NO: 5 (Genbank accession number NM_{—}000619) encoded by the nucleotide sequence of SEQ ID NO: 6 and fragments thereof. In addition, IFNγ encompasses polypeptides that have a high degree of similarity or a high degree of identity with the amino acid sequence of SEQ ID NO: 5 and which polypeptides are biologically active. According to an embodiment, IFNγ encompasses polypeptides substantially homologous to sequence of SEQ ID NO: 5, but which has at least one an amino acid sequence different from that of the original sequence because of one or more deletions, insertions or substitutions.

“Substantially homologous” means a variant amino acid sequence that is at least 80%, at least 85%, at least 90%, at least 95%, at least 96%, at least 97%, at least 98% or at least 99% identical to the original amino acid sequence, as disclosed above. The percent identity of two amino acid sequences can be determined by visual inspection and/or mathematical calculation, or more easily by comparing sequence information using known computer program used for sequence comparison such as BLAST (Basic Local Alignment Search Tool) or Clustal package version 1.83.

The term “IFNγ antagonist” or “IFNγ inhibitor” comprises all antagonists/inhibitors of all suitable forms of IFNγ or IFNγ receptors such as IFNγ receptor 1 and/or IFNγ receptor 2, or IFNγ effectors LIGHT, LT-βR, HVEM, or DcR3, or fragment thereof, described herein that antagonize one or more biological activity of IFNγ and/or of IFNγ receptors and/or of IFNγ variants or fragment thereof. For example, the IFNγ antagonists of the invention are able to antagonize the ability of IFNγ to interact with IFNγ receptor 1 and/or IFNγ receptor 2, to modulate the activation of the LIGHT-LT-βR death pathway (e.g. the ability to antagonize LIGHT expression) or the production of proinflammatory molecules through the IFNγ receptors complex. The term “IFNγ antagonist” includes but is not limited to: IFNγ antagonist specific antibodies of any sort (polyclonal, monoclonal, antibody fragments, antibody variants), chimeric proteins, natural or unnatural proteins with IFNγ antagonizing activities, small molecules, nucleic acid derived polymers (such as DNA and RNA aptamers, PNAs, or LNAs), peptidomimetics, fusion proteins, or gene therapy vectors driving the expression of such IFNγ antagonists, a viral vector encoding IFNγ antagonists. Further embodiments include as IFNγ antagonist, soluble IFNγ fusion proteins such as but not limited to molecules that would block intracellular signalization of IFNγ, soluble monomeric or oligomeric IFNγ receptor 1 and/or IFNγ receptor 2. Typically, IFNγ antagonists are able to bind IFNγ and/or to block the binding of IFNγ and other binding partners such as IFNγ receptors such as IFNγ 1 and/or IFNγ receptor 2.

The term “IFNγ aptamer” comprises non coding nucleic acid molecules which bind with high specificity and affinity IFNγ or IFNγ receptor 1 and/or IFNγ receptor 2, or IFNγ effectors LIGHT, LT-βR, HVEM, or DcR3, by adopting a specific secondary and tertiary structure. Aptamers may be selected from a group of sequences identified using SELEX™ (Systematic Evolution of Ligands by EXponential enrichment) method or a similar process (Cox et al., 1998, Biotechnol. Prog., 14, 845-850; Berezovski et al., 2006, J. Am. Chem. Soc., 128:, 1410-1411). IFNγ aptamers described herein are capable of specifically binding to and neutralizing IFNγ or IFNγ receptors, IFNγ receptor 1 and/or IFNγ receptor 2, or IFNγ effectors LIGHT, LT-βR, HVEM, or DcR3, thereby antagonizing one or more biological activity of IFNγ and/or of IFNγ receptors and/or of IFNγ variants or fragment thereof, or modulating the interaction between IFNγ and IFNγ receptor 1 and/or IFNγ receptor 2, or antagonizing the activation of the LIGHT-LT-βR death pathway (e.g. the ability to antagonize LIGHT expression) or the production of proinflammatory molecules through the IFNγ receptors complex. For example, IFNγ aptamers are generated via an iterative process of binding, partitioning, and amplification. Single-stranded DNA primers and templates are amplified into double-stranded transcribable templates by PCR. The library of sequences is either directly used for the selection of DNA aptamers, or transcribed to an RNA library for the selection of RNA aptamers. The template sequence may be for example a single stranded sequence composed of 40 random nucleotides (40N) flanked by defined primer-annealing sequences (5′-GGG AGG ACG AUG CGG [40N] CAG ACG ACU CGC CCG A-3′) (SEQ ID NO: 7) amplified with SELEX PCR primers 5′-TAA TAC GAC TCA CTA TAG GGA GGA CGA TGC GG-3′ (SEQ ID NO: 8) and 5′-TCG GGC GAG TCG TCT G-3′ (SEQ ID NO: 9). RNA molecules so generated are then screened for their ability to interact with human and/or murine IFNγ or IFNγ receptor 1 and/or IFNγ receptor 2, or IFNγ effectors LIGHT, LT-βR, HVEM, or DcR3, for example by affinity chromatography, magnetic bids, or filtration. Alternative rounds of selection and amplification are repeated under increasing stringency in order to generate a limited subset of highly specific, high-affinity candidates to the target molecule. Selected candidates may then be truncated and chemically modified to improve resistance to nucleases and pharmacological properties. IFNγ aptamers may comprise, but are not limited to, the oligonucleotide sequence 5′-GGG GTT GGT TGT GTT GGG TGT TGT GT-3′ (SEQ ID NO: 10) or fragments thereof which retain binding capability (Lee et al., 1996, Transplantation 62, 1297-1301; Ramanathan et al., 1994, J. Biol. Chem., 269, 24564-24574) or the oligonucleotide sequence 5′-CAG GUA AUU ACA UGA AGG UGG GUU AGG UAC UUU CAG GGU-3′ (SEQ ID NO: 11) or fragments thereof which retain binding capability (Kubik et al., 1997, J. Immunol., 159(1),:259-267). Aptamers may comprise biostable aptamers, such as Spiegelmers™ (Klussmann et al., 1996, Nat. Biotechnol., 14, 1112-1115; Vater and Klussmann, 2003, Curr. Opin. Drug. Discov. Devel., 6, 253-261).

The term “IFNγ chimeric protein” comprises, but is not limited to, molecularly, physically or chemically inactivated protein derivatives or fragments of IFNγ with preserved affinity to IFNγ receptor 1 and/or IFNγ receptor 2, or of IFNγ receptor 1 or IFNγ receptor 2 with preserved affinity to IFNγ, or of LT-βR with preserved affinity to LIGHT, or of HVEM with preserved affinity to LIGHT, or of DcR3 (nucleotide SEQ ID NO: 12 (Genbank accession number NM_{—}032945.2) and amino acid SEQ ID NO: 13) with preserved affinity to LIGHT and FasL. Such derivatives or fragments may be fused to an oligomerisation domain allowing clustering of the dominant negative such as COMP domain or Fc fragment of immunoglobulin (Holler et al., 2000, J. Immunol. Methods, 237, 159-173) or to a fragment of another human protein.

The term “viral vector” comprises recombinant adeno-associated virus (rAAV) vectors and recombinant lentiviral (rLV) vectors. In a particular embodiment, the term “viral vector comprises self-complementary adeno-associated virus (scAAV) vectors.

The term “peptidomimetic” is defined as a peptide analog containing non-peptidic structural elements, which peptide is capable of mimicking or antagonizing the biological action(s) of a natural parent peptide. A peptidomimetic does no longer have classical peptide characteristics such as enzymatically scissile peptide bonds.

The term “expression control element” encompasses a sequence which provides for transcription and translation of a gene and/or controls the expression of a protein for example in a desire host cell in vivo.

The term “neural cell” includes cells in the vicinity of motoneurons (upper and lower) such as for example astrocytes (typically when a method according to the invention comprises the delivery of a nucleic acid via systemic or intraspinal delivery) and other neuronal cell types such as motoneurons (typically when a method according to the invention comprises the delivery of a nucleic acid via intramuscular or systemic delivery), oligodendrocytes and interneurons.

The term “microglial cell” includes resting or infiltrating microglia.

The term “meningeal cell” includes membranes which envelop the central nervous system.

The expression “risk of developing a motoneuron disorder” refers to a higher risk of developing a motoneuron disorder than an individual (such as a mammal), who does not present elevated IFNγ levels.

### Compositions

IFNγ antagonists according to the invention may be administered as a pharmaceutical formulation which can contain one or more polypeptides according to the invention in any form described herein. Compositions of this invention may further comprise one or more pharmaceutically acceptable additional ingredient(s) such as alum, stabilizers, antimicrobial agents, buffers, coloring agents, flavoring agents, adjuvants, and the like.

IFNγ antagonists of the invention, together with a conventionally employed adjuvant, carrier, diluent or excipient may be placed separately into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) and transnasal use. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. IFNγ antagonist compositions according to the invention are preferably injectable.

IFNγ antagonists of this invention may also be liquid formulations including, but not limited to, aqueous or oily suspensions, solutions, emulsions, syrups, and elixirs. The IFNγ antagonists may also be formulated as a dry product for reconstitution with water or other suitable vehicle before use. Such liquid preparations may contain additives including, but not limited to, suspending agents, emulsifying agents, non-aqueous vehicles and preservatives. Suspending agent include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Injectable compositions are typically based upon injectable sterile saline or phosphate-buffered saline or other injectable carriers known in the art.

IFNγ antagonists of this invention may also be formulated for parenteral administration, including, but not limited to, by injection or continuous infusion. Typically, continuous infusion of IFNγ antagonists according to the invention may be achieved by implantation and or transplantation into the central nervous system of modified cells which express and secrete IFNγ antagonists, of encapsulated cells or through the administration via osmotic or mechanic pumps.

IFNγ antagonists of this invention may also be formulated for intranasal delivery, including, but not limited to, the use of specifically-adapted devices such as devices targeting upper third of the nasal cavity, fiber optic guided scopes or flexible nasopharyngoscopes that can spray the formulations directly on the roof of the nasal cavity, electronic atomizers, pressurized olfactory delivery systems, intranasal intubation, intranasal drops and mists, or locally implanted extended release devices. Typically, intranasal delivery methods target IFNγ antagonists directly to the brain from the nasal mucosa, while minimizing delivery to the blood, thereby avoiding metabolism and elimination by the liver and kidneys, binding by plasma proteins, and also unwanted systemic exposure and side-effects.

IFNγ antagonists of this invention may also be formulated as a depot preparation, which may be administered through implantation or intramuscular, or intravenous, or intracerebroventricular, or intrathecal or intracisternal, or intraperitoneal, or subcutaneous, or intranasal, or intravitreal, or transcleral, or epidural, or oral administration. IFNγ antagonists of this invention can also be administered in sustained release forms or in sustained release drug delivery systems. Further materials as well as formulation processing techniques and the like are set out in Part 5 of Remington's Pharmaceutical Sciences, 21^{st} Edition, 2005, University of the Sciences in Philadelphia, is Lippincott Williams & Wilkins, which is incorporated herein by reference.

IFNγ antagonists of this invention may also be administered as a non-replicative viral vector.

According to a particular embodiment, a viral vector according to the invention comprises recombinant adeno-associated virus (rAAV) vector and recombinant lentiviral (rLV) vector, comprising at least one expression control element operably linked to a nucleic acid sequence encoding for an IFNγ antagonist. In a particular embodiment, the IFNγ antagonist is secreted by astrocytes.

In another particular embodiment, a viral vector according to the invention comprises self-complementary adeno-associated viral vectors (scAAV), comprising a single-stranded inverted-repeat genome separated by a mutated terminal resolution site designed to allow DNA can fold on itself and produce a double stranded DNA without requiring DNA synthesis (McCarty et al., 2003, Gene Ther., 10, 2112-2118). ScAAV vectors have improved transduction efficiency and stronger transgene expression in several tissues, including the central nervous system, bone marrow, and muscle.

In a particular embodiment, the viral vector according to the invention is able to transduce cells selected from neural, microglial and meningeal cells throughout the brain and spinal cord.

In a particular embodiment, the invention provides a viral vector comprising at least one expression control element operably linked to a nucleic acid sequence encoding for an IFNγ antagonist, wherein the nucleic acid sequence encoding for an IFNγ antagonist comprises a nucleic acid sequence encoding for IFNγR1 (SEQ ID NO: 2) and a nucleic acid sequence encoding for an oligomerisation domain (SEQ ID NO: 19).

In a further particular embodiment, the invention provides a viral vector according to the invention wherein the nucleic acid sequence encoding for an IFNγ antagonist encodes for a mouse IFNγR1-COMP (SEQ ID NO: 15) or a variant thereof being at least 80% identical to SEQ ID NO: 15, or for a human IFNγR1-COMP (SEQ ID NO: 27) or a variant thereof being at least 80% identical to SEQ ID NO: 27.

In another further particular embodiment, the invention provides a viral vector according to the invention wherein the nucleic acid sequence encoding for an IFNγ antagonist encodes for human IFNγR1-COMP and has a sequence consisting of SEQ ID NO: 26, or for mouse IFNγR1-COMP and has a sequence consisting of SEQ ID NO: 14.

In another particular embodiment, the invention provides a viral vector comprising at least one expression control element operably linked to a nucleic acid sequence encoding for an IFNγ antagonist, wherein the nucleic acid sequence encoding for an IFNγ antagonist comprises a nucleic acid sequence encoding for DcR3 (SEQ ID NO: 13) and a nucleic acid sequence encoding for an oligomerisation domain (SEQ ID NO: 19).

In a further particular embodiment, the invention provides a viral vector according to the invention wherein the nucleic acid sequence encoding for an IFNγ antagonist encodes for DcR3-COMP (SEQ ID NO: 17) or a variant thereof being at least 80% identical to SEQ ID NO: 17.

In another further particular embodiment, the invention provides a viral vector according to the invention wherein the nucleic acid sequence encoding for an IFNγ antagonist encodes for DcR3-COMP and has a sequence consisting of SEQ ID NO: 16.

In a particular aspect, an AAV vector according to the invention comprises an AAV serotype 9 or an AAV serotype 6, e.g. the AAV vector according to the invention comprises capsid proteins of AAV serotype 9 or 6. In another aspect, an AAV vector according to the invention comprises AAV serotype 6, in particular as vehicle to transduce motoneurons following intramuscular administration. In another aspect, an AAV vector according to the invention comprises AAV serotype 9, in particular as vehicle to transduce astrocytes (eventually throughout the brain and the spinal cord) following intravenous administration.

In a particular embodiment, a viral vector according to the invention is able to drive expression of an IFNγ antagonist together with an oligomerisation domain, whereby the oligomerisation of the IFNγ receptor and thereby of the secreted IFNγ antagonist is favoured. Typically, such viral vector includes a nucleic acid sequence encoding for an oligomerisation domain allowing clustering of the dominant negative such as COMP domain or Fc fragment of immunoglobulin as described in Holler et al., 2000, above. In a further particular embodiment, a viral vector according to the invention includes a nucleic acid sequence encoding for an oligomerisation domain substantially homologous to sequence of SEQ ID NO: 19, but which has at least one an amino acid sequence different from that of the original sequence because of one or more deletions, insertions or substitutions. For example, the invention includes a nucleic acid sequence encoding for a variant of an oligomerisation domain of SEQ ID NO: 19, being at least 80% identical to SEQ ID NO: 19.

In a further particular embodiment, the IFNγ antagonist is an IFNγ antibody or IFNγ receptor antibody such as IFNγ receptor 1 and/or IFNγ receptor 2 antibodies or fragment thereof.

In another further embodiment, the IFNγ antagonist is an IFNγ antibody.

In another further embodiment, IFNγ antagonist is a viral vector.

In another particular embodiment, the IFNγ antagonist is a viral vector according to the invention.

In another further particular embodiment, is provided a viral vector comprising at least one expression control element operably linked to a nucleic acid sequence encoding for an IFNγ antagonist, wherein the nucleic acid sequence encoding for an IFNγ antagonist comprises a nucleic acid sequence encoding for IFNγR1 and a nucleic acid sequence encoding for an oligomerisation domain. According to another further particular embodiment, is provided a viral vector comprising at least one expression control element operably linked to a nucleic acid sequence encoding for an IFNγ antagonist, wherein the nucleic acid sequence encoding for an IFNγ antagonist comprises a nucleic acid sequence encoding for DcR3 and a nucleic acid sequence encoding for an oligomerisation domain.

In another further embodiment, is provided a viral vector according to the invention wherein expression control element is selected from neuronal or glial-specific promoters (e.g. GfaABC1-D).

In another particular embodiment, is provided a viral vector according to the invention for use as a medicament.

In another particular embodiment, is provided a viral vector according to the invention for the treatment of a motoneuron disease or disorder such as an ALS disorder.

In another particular embodiment, is provided a pharmaceutical preparation comprising at least one viral vector according to the invention and pharmaceutically acceptable carrier or excipient.

In another particular embodiment, is provided a use of a viral vector according to the invention for the manufacture of a medicament for the treatment of a motoneuron disorder.

In a further particular embodiment is provided a use according to the invention, wherein the rAAV vector is purified from contaminating helper adenovirus so that the IFNγ antagonist is expressed in the absence of a destructive immune response to the IFNγ antagonist. For example, rAAV may be purified by iodixanol or cesium chloride gradient; heparin or mucin columns; by high-pressure liquid chromatography (HPLC) on heparin columns or by ion exchange HPLC following the procedure described in Grieger et al., 2006, Nat. Protoc., 1(3): 1412-28; Towne et al., 2008, Mol. Ther. 16, 1018-25; Kaludov et al., 2002, Hum. Mol. Genet., 1; 13(10): 1235-43. Vector preparations procedures for the large-scale production of viral vectors may be carried out via, but not limited to, the utilization of bioreactors and based on the baculovirus/insect cell system (Virag et al., 2009, Hum. Gene Ther., 20(8), 807-17).

In a further particular embodiment is provided a use according to the invention, wherein the lentiviral vector is a replication-defective lentiviral vector modified to increase transgene expression, produced in 293T cells, concentrated by ultracentrifugation and resuspended in phosphate-buffered saline (PBS)/1% bovine serum albumin (BSA) (Hottinger et al., 2000, J. Neurosci., 20:5587-93).

In another further particular embodiment is provided a use according to the invention wherein the medicament is in a form adapted for delivery of the viral vector by intramuscular, or intravenous, or intracerebroventricular, or intrathecal or intracisternal, or intraperitoneal, or subcutaneous, or intranasal, or intravitreal, or transcleral, or epidural, or oral administration, where the said IFNγ antagonist is expressed.

In another particular aspect, the medicament is adapted for delivery by single or repeated administration.

In a particular aspect, the medicament comprises at least 10⁵ transducing unit of rAAV.

In another embodiment, is provided a method of treating a motoneuron disease or disorder in a subject in need thereof, comprising administering in said subject a pharmaceutical composition which comprises an IFNγ antagonist according to the invention.

In another particular embodiment, is provided an IFNγ antibody for the treatment of a motoneuron disease or disorder.

In another particular embodiment, is provided a use of an IFNγ antibody for the preparation of a pharmaceutical preparation for the treatment of a motoneuron disease or disorder.

In another particular embodiment, is provided a method for delivering a nucleic acid sequence encoding an IFNγ antagonist to cells selected from neural, microglial and meningeal cells comprising: (a) providing a virion comprising a viral vector, said vector comprising at least one expression control element operably linked to a nucleic acid sequence encoding for an IFNγ antagonist; (b) bringing the virion into contact with said cells, whereby transduction of the viral vector results in the expression of said nucleic acid sequence in the transduced cells and the expression of said nucleic acid sequence by said cells.

In a further particular embodiment, is provided a method according to the invention, wherein expression of said nucleic acid sequence occurs in the transduced astrocyte cells and the expression of said nucleic acid sequence by astrocyte cells result in the reduction of motoneuron damage.

In another further particular embodiment, is provided a method of treating a motoneuron disorder in a subject in need thereof, comprising administering in said subject a pharmaceutical composition which comprises (a) a pharmaceutically acceptable excipient; and (b) virions comprising a viral vector, said viral vector comprising a nucleic acid sequence encoding for an IFNγ antagonist, operably linked to at least one expression control element that controls expression of the said IFNγ antagonist.

In a further particular embodiment, is provided a viral vector, a use or a method according to the invention, wherein the IFNγ antagonist is an IFNγ antibody or IFNγ receptor antibody such as IFNγ receptor 1 and/or IFNγ receptor 2 antibodies or fragment thereof.

In a further particular embodiment, is provided a viral vector, a use or a method according to the invention, wherein the IFNγ antagonist is a viral vector according to the invention.

In another aspect, the invention provides a method of treating a motoneuron disease or disorder in a subject in need thereof, comprising implanting and/or transplanting genetically engineered stem cells secreting an IFNγ antagonist in the central nervous system of said subject. In particular, the method comprises steps described in Suzuki et al., 2008, Mol. Ther., 16(12):2002-10.

In another aspect, the invention provides an in vitro method for detection and/or prognosis of a motoneuron disease in a sample from a subject, comprising the following steps: (a) measuring IFNγ levels in a sample from said subject; and (b) comparing IFNγ levels data obtained in step (a) to IFNγ level data of patients suffering from a motoneuron disease, wherein IFNγ levels correlate with a motoneuron disease status in said subject. Typically, the sample can be a blood sample, a cerebrospinal fluid, or a tear sample. IFNγ levels may be measured by various techniques such as immunoassays, such as ELISA, immunoblots, and lateral flow. Typically, IFNγ levels higher than about 100 pg/ml is an indication that the subject is suffering from or is at risk of developing a motoneuron disease and IFNγ levels lower than about 10 pg/ml is an indication that the subject is not suffering from or not likely to develop a motoneuron disease.

In a further aspect, the invention provides an in vitro method according to the invention wherein the sample is selected from a serum sample, a cerebrospinal fluid sample and a tear sample. Methods considered are e.g. ELISA, RIA, EIA, mass spectrometry, microarray analysis, ELISPOT, flow cytometry, bead-based assay, PCR, RT-PCR, immuno-PCR techniques, or other high-sensitivity immunoassay detection methods such as radioimmunoassay. Typically an in vitro method according to the invention is based on an ELISA assay according to known methods. For example, a microtiter plate is coated with one type of antibody directed against IFNγ, then the plate is blocked and a sample or a standard is loaded on the said plate, then a second type of antibody against IFNγ is applied, a third antibody detecting the particular type of the second antibody conjugated with a suitable label is then added, and the label is used to quantify the amount of IFNγ.

In a further aspect, the invention provides a method according to the invention wherein the subject is a mammal, typically a human. According to another further aspect, the invention provides a method according to the invention wherein the subject is an animal model of a motoneuron disease such as a rat or a mouse.

In another aspect, the invention provides a kit for in vitro detecting a motoneuron disease in a subject comprising: (a) at least one sample testing device that provides a readable signal proportional to the IFNγ concentration in a sample; (b) an electronic monitor having reading means to read the readable signal obtained under step (a) and incorporating computer means to interpret the readable signals and to determine therefrom in conjunction with data from previous sample tests a motoneuron status of said subject. Typically an in vitro method and a kit according to the invention have the advantage to detect motoneuron degenerescence via IFNγ level data at disease onset and symptomatic stage of the motoneuron disorder.

In a particular embodiment, a motoneuron disorder is an ALS disorder.

### Mode of Administration

IFNγ antagonists of this invention may be administered in any manner including, but not limited to, intravenous, intra-arterial, intraperitoneal, subcutaneous, intramuscular, intracerebroventricular, intracisternal andintrathecal. In a further aspect, IFNγ antagonists may be administered via intranasal, intravitreal, transcleral, epidural, and oral administration. The IFNγ antagonists of this invention may also be administered in the form of an implant, which allows slow release of the compositions as well as a slow controlled i.v. infusion.

In particular, IFNγ antagonists according to the invention may be delivered as in the intramuscular space such as hindlimb, forelimb, dorsal and facial muscles or muscles of the trunk or spinal cord (intraparenchymal), cisterna magna, intrathecal space, nasal mucosa, to obviate systemic delivery and improve their half-life, their improve access to the spinal cord and brainstem and brain system or decrease potentiel side effects.

The examples illustrating the invention are not intended to limit the scope of the invention in any way. The dosage administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired.

According to one aspect, a treatment according to the invention comprises either administering an effective amount of nucleic acid molecules directly binding with high specificity and affinity IFNγ, or IFNγ receptor 1 and/or IFNγ receptor 2, or IFNγ effectors LIGHT, LT-βR, HVEM, or DcR3 or using a viral vector encoding an IFNγ antagonist such as described herein.

### Combination

According to the invention, an IFNγ antagonist, such as an IFNγ antibody, IFNγ aptamer, IFNγ chimeric protein, or a viral vector according to the invention and pharmaceutical formulations thereof can be administered alone or in combination with a co-agent useful in the treatment of a motoneuron disorder such as ALS disorders, e.g. for example Riluzole.

The invention encompasses the administration of a IFNγ antagonist according to the invention, such as an IFNγ antibody, IFNγ aptamer, IFNγ chimeric protein, or a viral vector according to the invention, or pharmaceutical formulations thereof, wherein the IFNγ antagonist or the pharmaceutical formulation thereof is administered to an individual prior to, simultaneously or sequentially with other therapeutic regimens or co-agents useful in the treatment of an ALS disorder (e.g. multiple drug regimens), in a therapeutically effective amount. IFNγ antagonist according to the invention or the pharmaceutical formulations thereof that are administered simultaneously with said co-agents can be administered in the same or different composition(s) and by the same or different route(s) of administration.

### Patients

In an embodiment, patients according to the invention are patients suffering from a motoneuron disorder.

In a particular embodiment, patients according to the invention are suffering from an ALS disorder.

In a particular embodiment, patients according to the invention are suffering from sporadic and familial ALS, atypical ALS (extrapyramidal signs such as tremors), dementia in association with the classical phenotype of ALS including FTD-ALS (frontotemporal dementia ALS), i.e. cognitive impairment, and other motoneuron diseases such as spinal muscular atrophy (SMA), Kennedy's disease (or spinobulbar muscular atrophy), hereditary spastic paraplegia, Primary lateral sclerosis, progressive muscular atrophy.

References cited herein are hereby incorporated by reference in their entirety. The present invention is not to be limited in scope by the specific embodiments described herein, which are intended as single illustrations of individual aspects of the invention, and functionally equivalent methods and components are within the scope of the invention. The invention having been described, the following examples are presented by way of illustration, and not limitation.

### EXAMPLES

The following abbreviations refer respectively to the definitions below:

h (hour), i.c.v. (intracerebroventricular), AAV (adeno-associated viral), cDNA (complementary DNA), EDTA (ethylenediaminetetraacetic acid), GFAP (Glial fibrillary acidic protein), HEPES (N-2-hydroxyethylpiperazine-N′-2-ethanesulfonic acid), IU (International Unit), PCR (Polymerase Chain Reaction), TU (transduction unit).

### General Procedures & Conditions

In a particular aspect, the present invention consists of administering an IFNγ inhibitor according to the invention or inhibiting LIGHT-LT-βR pathway via IFNγ antagonists such as a function-blocking anti-IFNγ antibody or a non-replicative viral vector mediating the expression of IFNγ antagonists, injected via systemic, the intramuscular, intranasal, intrathecal, intracisternal or intracerebroventricular route.

### Example 1

### IFNγ-Induced Motoneuron Death

In order to support the role of IFNγ in motoneuron death in ALS, the following preliminary tests were conducted.

### In Healthy Motoneurons

IFNγ receptor chain 1 (IFNγR1) and chain 2 (IFNγR2) are expressed in nearly all Hb9::GFP motoneurons (embryonic motoneurons (E12.5) isolated from transgenic mice expressing the green fluorescent protein (GFP) under the control of the motoneuron-selective Hb9 promoter (Hb9::GFP) to facilitate motoneuron tracing) in vitro as shown by immunostaining 24 hours after seeding with antibodies directed against IFNγR1 or IFNγR2 as described below.

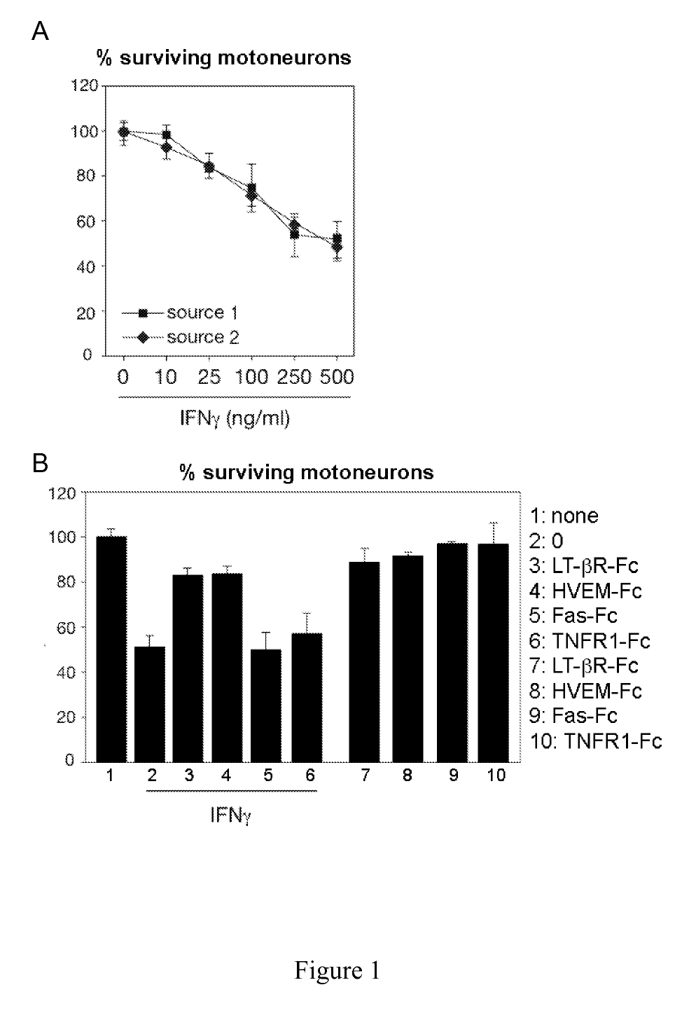

The exposure of motoneurons to a suboptimal dose of sLIGHT and increasing doses of IFNγ shows that IFNγ substantially enhances the LIGHT killing effect and that this synergetic lethal effect is specific to LIGHT since IFNγ had no effect on sFasL-induced death (FIG. 1B). Recombinant IFNγ induced death of about 50% of motoneurons in a dose-dependent manner (FIG. 1A). The synergistic lethal effect of IFNγ on LIGHT-induced death is restricted to motoneurons since neither cortical, hippocampal, sensory nor striatal neurons (all expressing both IFNγ receptors, IFNγR1 and IFNγR2) are sensitive to the combination of IFNγ with sLIGHT which is lethal to motoneurons.

IFNγ operates as a neuromodulatory cytokine by significantly enhancing expression levels of LIGHT. Further, it is observed that IFNγ is able to render freshly isolated motoneurons (which are not competent to die through LIGHT) responsive to sLIGHT or agonistic anti-LT-βR antibodies (functional goat polyclonal antibodies (R&D systems)) as shown by treating motoneurons or not at 0, 24, or 72 h with sLIGHT agonistic anti-LT-βR antibodies, IFNγ, or with IFNγ combined with either sLIGHT or anti-LT-βR antibodies.

Altogether, results support the role of IFNγ in specifically triggering a caspase-9 and -6 dependent death program in motoneurons through the LIGHT-LTβR pathway.

### In SOD1^{G93A} Mutant Motoneurons and Astrocytes

Motoneurons from mice over expressing the G93A SOD1 mutation (mice developing a motor syndrome with features of the human ALS as described in Gurney et al., 1994, Science, 264, 1772-1775) were cultured as previously described (Raoul et al., 2002, Neuron, 35, 1067-1083). Comparison of their susceptibility to IFNγ and LIGHT to wild-type motoneurons shows that SOD1^{G93A} mutation does not exacerbate the responsiveness of motoneurons to IFNγ and LIGHT.

The expression levels of IFNγ and of LIGHT were measured in SOD1^{G93A} rat astrocytes by immunoblotting on total protein extracts, using anti-IFNγ and anti-LIGHT antibodies: in contrast to the wild-type, SOD1^{G93A} astrocytes expressed substantial levels of IFNγ but no difference between mutant and wild-type astrocytes was observed with respect to expression levels of LIGHT.

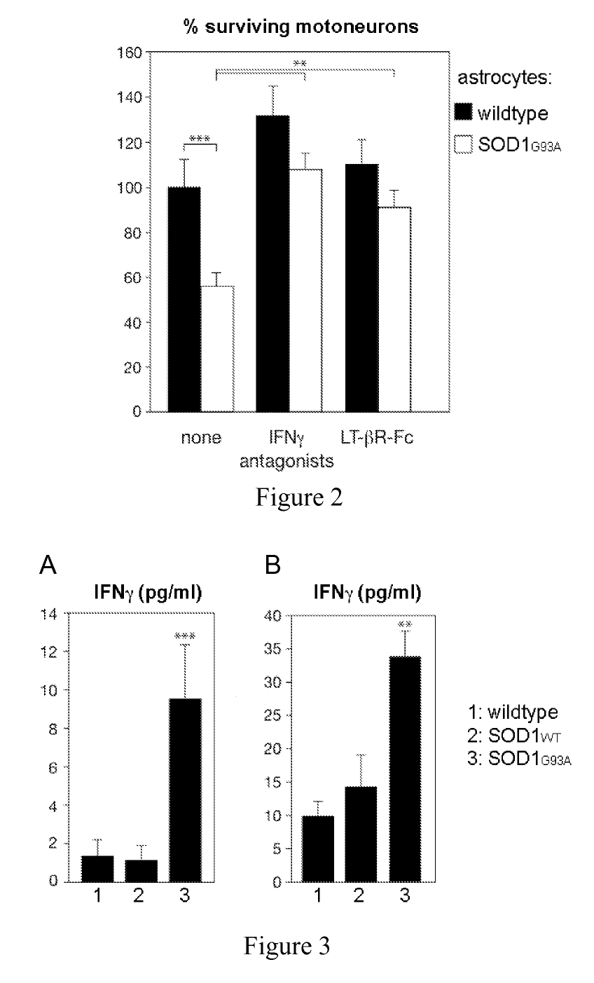

In a well-characterized co-culture system of immune purified wild-type rat motoneurons and wildtype or SOD1^{G93A} rat astrocytes monolayers (Cassina et al., 2008, J. Neurosci., 28, 4115-4122) (same responsivity of purified rat motoneurons to sLIGHT, agonistic anti-LT-βR antibodies (R&D systems) and IFNγ as mouse motoneurons was checked) the vulnerability of motoneurons to astrocytes expressing mutated SOD1 was confirmed by counting phase-bright neurons using morphological criteria as previously described (Raoul et al., 1999, J. Cell. Biol., 147, 1049-1062): after 48 h of co-culture, about 50% of the purified wild-type motoneurons plated on the SOD 1^{G93A} astrocytes died compared to motoneurons cultured on wild-type astrocytes (FIG. 2).

Collectively, these results support that an IFNγ-induced motoneuron selective death is involved in the astrocytic neurotoxicity conferred by mutant SOD1.

### Example 2

### IFNγ Levels in Serum and Cerebrospinal Fluid are Increased in ALS

The efficacy of a method for detection and/or prognosis of ALS and disease progression according to the invention are tested through the measure of IFNγ levels in serum and spinal cord of SOD1^{G93A} mice and ALS patients, at different disease stages.

### IFNγ Levels in Serum and Lumbar Spinal Cord of Sod1^{G93A} Mice Compared to Age-Matched Wildtype and SOD1^{WT} Mice

An enzyme-linked immunoabsorbent (ELISA) analysis was carried out to titer for IFNγ levels wherein mice of indicated genotype were bled at 13 weeks of age. Lumbar spinal cords were dissociated in 50 mM Tris-Hcl pH 7.5, 100 mM NaCl containing a cocktail of protease inhibitor (complete EDTA-free tabs, Roche Diagnostic). Spinal cord homogenates were centrifuged at 10,000×g for 10 min at +4° C. and protein concentration was determined in supernatants using Bradford assay (BioRad). Levels of IFNγ were then determined using mouse IFNγ ELISA kit II OptEIA™ (BD Biosciences) according to manufacturer's recommendations (BD Biosciences). IFNγ levels were shown to be increased significantly in serum and lumbar spinal of SOD1^{G93A} mice compared to age-matched wildtype and SOD1^{WT} mice (FIGS. 3A & B).

### At Different Disease Stages

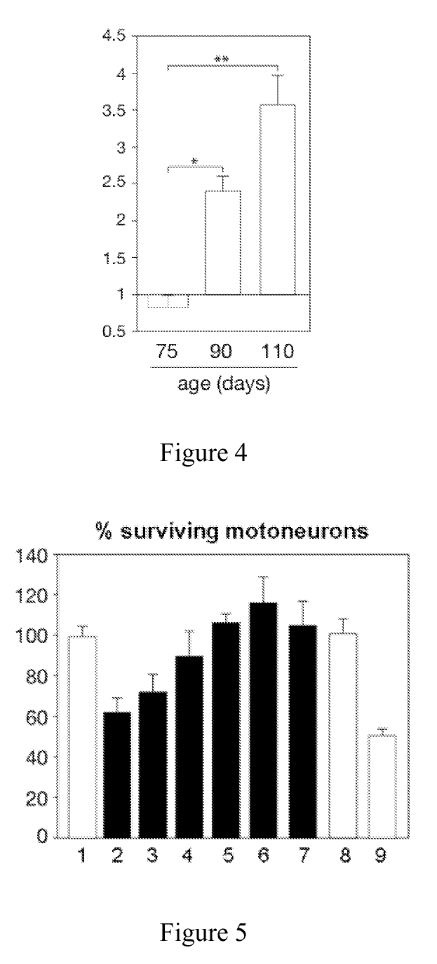

IFNγ expression was monitored during the course of the disease in wildtype and SOD1^{G93A} mice by quantitative analysis of total protein extracts from lumbar spinal cords and immunohistological analyses. Whereas total levels of IFNγ in the spinal cord of SOD1^{G93A} were barely detectable at pre-symptomatic stage and indistinguishable from those seen in non-transgenic mice, levels significantly increased at early disease onset and were further enhanced at symptomatic stages compared to wild-type littermate control mice (FIG. 4) and IFNγ is shown to be specifically expressed by astrocytes. IFNγ was not detectable in the spinal cord of both wild-type and pre-symptomatic mutant SOD1 mice but, readily detected in astrocytes at early onset and symptomatic stages as identified with glial fibrillary acidic protein (GFAP) antibodies (Millipore), but not by in microglial cells identified with the ionized calcium binding adaptor molecule 1 (Iba1) antibody (Wako Chem Ind). The only other cell type positive for IFNγ at early onset and symptomatic stages of the disease were motoneurons, as identified using non-phosphorylated neurofilament (SMI32) and VAChT antibodies (Steinberger Monoclonals).

Consistent with the increase of total IFNγ expression and its astrocytic localization at early onset and symptomatic stages, the percentage of motoneurons immunoreactive for IFNγ increased significantly at 13 and 16 weeks of age compared to 10.5-week-old SOD1^{G93A} mice and age-matched wild-type mice. A similar increase in IFNγ was observed in symptomatic (52 weeks) SOD1^{G85R} transgenic mice (mice developing a motor syndrome with features of the human ALS as described in Bruijn et al., 1997, Neuron, 18, 327-338), whereas mice overexpressing non-pathogenic SOD1^{WT} (Reaume et al., 1996, Nat. Genet., 13, 43-47) did not show any increase in IFNγ both in motoneurons and glial cells and were indistinguishable from the age-matched non-transgenic mice.

In summary, these results show that expression of IFNγ by mutant astrocytes and motoneurons occurs at onset and symptomatic stages and suggest that IFNγ potentially contributes to the progression of motoneuron disease beyond than the time of onset.

### Levels of IFNγ Increase in Human ALS

Expression levels of IFNγ in postmortem spinal cord samples of sporadic ALS patients and non-ALS controls were investigated by Western Blot analyses. Densitometric analysis using the Image Processing and Analysis in Java (ImageJ) software showed an elevation of IFNγ levels in spinal cords of human ALS compared to controls. Furthermore, immunohistological analyses of the expression pattern of IFNγ, LT-βR and LIGHT as described below in ALS spinal cord sections revealed significant staining for IFNγ in ventral horn motoneurons as well as in numerous surrounding glials cells in ALS patient but not in control tissues. Coherently with results in mice, both LT-βR and LIGHT were mainly expressed in motoneurons in ALS and non-ALS tissues. These observations in sporadic ALS spinal cords provide strong evidence for the implication of IFNγ and its effector pathway in the disease. IFNγ levels in the cerebrospinal fluid, blood, spinal cord and tears samples from both sporadic and familial ALS patients are studied by Western blot, ELISA, cytometry and quantitative PCR and lateral flow. For each patient, blood samples will be collected in dry tube for serum, EDTA tube for flow cytometry and BD Vacutainer® CPT™ Cell Preparation Tube with Sodium Citrate for plasma and isolation of PBMC which will be frozen in nitrogen liquid quantitative PCR. CSF samples will be immediately centrifuged at 800 r.p.m for 5 min. The cell pellet will be analyzed by flow cytometry. The liquid phase of CSF is stored at −80° C. until cytokine assay. The levels of IFNγ in the serum and CSF samples will be studied by a cytokine bead array (CBA, BD bioscience). A phenotypic analysis on whole blood sample using multicolour flow cytometry with the following makers IFNγ, NKp46, CD3, CD4, CD8, CD56, CD69, CD11c and CD14.

### Cell Cultures

Motoneurons from E12.5 spinal cord of CD1, Hb9::GFP, or SOD1^{G93A} embryos were isolated as described (Arce et al., 1999, J. Neurosci. Res., 55, 119-12) modified by Raoul et al., 2002, above, using iodixanol density gradient centrifugation. Motoneurons were plated on poly-ornithine/laminin-treated wells in the presence (or not when mentioned) of a cocktail of neurotrophic factors (0.1 ng/ml glial-derived neurotrophic factor (GDNF), 1 ng/ml brain-derived neurotrophic factor (BDNF), and 10 ng/ml ciliary neurotrophic factor (CNTF) in supplemented Neurobasal Medium™ (Invitrogen). When needed, motoneurons were electroporated before plating with the indicated expression constructs as previously described in Raoul et al., 2002, above. Motoneurons from E14 rat embryos were immunopurified using Ig192 mouse monoclonal anti-p75 antibody as previously described in Cassina et al., 2008, above and cultured in supplemented Neurobasal medium in the presence of GDNF (0.1 ng/ml). Cortical, hippocampal, dorsal root ganglion neurons and striatal neurons were isolated from E17.5 embryos as described in Zala et al., 2005, Neurobiol. Dis., 20, 785-798 and Raoul et al., 2002, above. Cortical, hippocampal, sensory and striatal neurons were plated on poly-ornithine/laminin-treated wells and cultured in Neurobasal medium complemented with 1 mM sodium pyruvate, 2% B27 supplement (Invitrogen) at the exception of sensory neurons that were maintained in the same supplemented Neurobasal medium used for motoneurons but in the presence of 100 ng/ml nerve growth factor (NGF) instead of GDNF, BDNF and CNTF. Unless otherwise indicated, cell survival experiments were done on neurons isolated from CD1 mice. All neuronal types were seeded at the density of 1,500 cells/cm² and surviving neurons were directly counted under light or fluorescence microscopy. Cos-7 cells were maintained in Dulbecco Modified Eagle Medium (DMEM, Invitrogen) supplemented with 10% fetal bovine serum. For expression analysis of FLAG tagged LT-βR (expression vector LT-bR ou TNFRSF3: Genbank accession number NM_{—}002342) and HA tagged LIGHT (expression vector LIGHT ou TNFSF14: Genbank accession number AF036581), cells were transfected using Fugene 6 following manufacturer's instruction (Roche diagnostics). Cos-7 transfected with pcDNA3.1 mammalian expression vector encoding for human LIGHT and human LT-bR (Aebischer et al., 2010, Cell Death Differ., November 12) were used as positive control for immunoblot detection of LIGHT and LT-bR.

### Animals

All animal experiments were done in compliance with the European Community and National directives for the care and use of laboratory animals. HB9::GFP mice (Wichterley et al. 2002, Cell, 110, 385-397) were maintained on a CD1 background. SOD1^{G93A} mice were maintained on a mixed B6SJL background (Gurney et al., 1994, above). SOD1^{G85R} (Bruijn et al., 1997, above) and SOD1^{WT} (Reaume et al., 1996, above) mice were maintained on a C57BL/6 background (Bruijn et al., 1997, Annu. Rev. Neurosci., 27, 723-749). Sprague-Dawley SOD1^{G93A} L26H rats were maintained as described in Cassina et al., 2008, above.

### Proteins and Chemicals for Survival Assay

Soluble human recombinant LIGHT and FasL, enhancer antibodies used for aggregating tagged sFasL, Fas-Fc, TNFR1-Fc, LT-βR-Fc, were purchased from Alexis Biochemicals. Soluble mouse recombinant LIGHT, functional goat polyclonal anti-LT-βR anti-HVEM antibodies were purchased from R&D systems. Soluble mouse recombinant IFNγ (source 1) was purchased from Calbiochem. Soluble mouse recombinant IFNγ (source 2) and soluble recombinant rat IFNγ were from PBL Biomedical laboratories. Neutralizing goat polyclonal anti-IFNγ antibodies (15027) were purchased from Sigma-Aldrich.

### Immunocytochemistry

Hb9::GFP motoneurons were purified from E12.5 HB9::GFP embryos (Wichterley et al. 2002, above) and seeded on poly-ornithine/laminin-treated glass coverslips at the density of 5,000 cells/cm² and cultured in the supplemented Neurobasal medium as above. At indicated time, neurons were processed for immunocytochemistry as previously described in Raoul et al., 2005, Nat. Med., 11, 423-428. Primary antibodies were: anti-LT-βR (sc-8376, Santa Cruz Biotechnology, 1:50), anti-HVEM (AF2516, R&D systems, 1:50), anti-LIGHT (sc-28880, Santa Cruz Biotechnology, 1:50), anti-IFNγR1 (559911, BD Biosciences, 1:250), anti-IFNγR2 (ab31606, Abcam, 1:2000). Alexa Fluor 555-conjugated donkey anti-goat, anti-rabbit or anti-mouse were used as secondary antibodies (Invitrogen). Images were taken using a Zeiss LSM510 laser scanning confocal microscope, manufactured by Carl Zeiss (Jena, Germany).

### Western Blot

Neurons were plated at the density of 20,000 cells/cm² in 6-cm diameter dishes containing corresponding complemented Neurobasal medium (see above). Sodium dodecyl sulfate polyacrylamide gel electrophoresis (SDS-PAGE) and Western blotting were carried out on neurons, astrocytes (duplicates) and dissected lumbar spinal cords using protocol previously described in Raoul et al., 2005, above. Primary antibodies were anti-LT-βR (sc-8377, Santa Cruz Biotechnology, 1:500), anti-LIGHT (sc-28880, Santa Cruz Biotechnology, 1:500), anti-IFNγ (sc-52557, Santa Cruz Biotechnology, 1:500), anti-IFNγR1 (559911, BD Biosciences, 1:500), anti-IFNγR2 (ab31606, Abcam, 1:2000), anti-α-tubulin (B-5-1-2, Sigma-Aldrich, 1:20,000), anti-actin (AC-40, Sigma-Aldrich, 1:20,000). Proteins were detected using horseradish peroxidase (HRP)-conjugated secondary antibodies and visualized with the chemiluminescent HRP substrate (Millipore). Where indicated, immunoblot images were quantified and normalized relative to the α-tubulin or actin levels using the NIH ImageJ software.

### Motoneuron-Astrocyte Cocultures

Primary astrocyte cultures were prepared from spinal cord of P1-P2 wildtype and SOD1^{G93A} rats as previously described (Cassina et al., 2008, above). Astrocytes were plated at a density of 2×10⁴ cells/cm² and maintained in DMEM supplemented with 10% fetal bovine serum, HEPES (3.6 g/l), penicillin (100 IU/ml) and streptomycin (100 μg/ml). Astrocyte monolayers were 98% pure as determined by GFAP immunoreactivity and devoid of OX42-positive microglial cells. Wildtype motoneurons, purified as above, were plated on rat astrocyte monolayer of different genotypes at the density of 300 cells/cm² and maintained for 48 h in L15 medium (Invitrogen) supplemented with 2% horse serum, 0.63 mg/ml bicarbonate, 5 μg/ml insulin, 0.1 mg/ml conalbumin, 0.1 mM putrescine, 30 nM sodium selenite, 20 nM progesterone, 20 mM glucose, 100 U/ml penicillin, 100 μg/ml streptomycin.

### Immunohistochemistry

Immunostaining of lumbar spinal cord sections was performed as described previously by Raoul et al., 2006, Proc. Natl. Acad. Sci. U.S.A., 103, 6007-6012. The following antibodies were used: anti-IFNγ (15027, Sigma-Aldrich, 1:100), anti-non-phosphorylated neurofilament (SMI32, Sternberger Monoclonals, 1:500), anti-GFAP (MAB360, Millipore, 1:500), anti-ionized calcium binding adaptor molecule 1 (Iba1, Wako Chemical Industries, 1:100), anti-LT-βR (sc-8376, Santa Cruz Biotechnology, 1:50), anti-LIGHT (sc-28880, Santa Cruz Biotechnology, 1:50) and anti-VAChT (V5387, Sigma-Aldrich, 1:2500). Proteins were detected using either fluorochrome-conjugated secondary antibodies (Alexa Fluor 488 or 555) or the peroxidase/DAB detection system following the manufacturer's instruction (Dako).

### Statistical Analysis

Statistical significance was determined by unpaired two-tailed t test or, when indicated, by an one-way analysis of variance (ANOVA) followed by a Tukey-Kramer's post hoc test using the GraphPad Instat software. Significance was accepted at the level of P<0.05.

Altogether those results show that astrocytes expressing ALS-linked mutant SOD1 mediate the selective death of motoneurons through the proinflammatory cytokine IFNγ, which activates the LIGHT-LT-βR death pathway.

### Example 3

### Rescue from Neurotoxicity of Astrocytes by Antagonizing the IFNγ-Induced LIGHT-Triggered Death Pathway

The efficacy of a method according to the invention are tested through the therapeutic impact of the inhibition of IFNγ, by using an antagonizing antibody in vitro and in vivo. Disease progression and lifespan is determined by behavioural and histopathological analysis as described in Example 2.

Purified Motoneurons Rescued with Neutralizing Anti-IFNγ Antibodies

The potential of anti-IFNγ antibodies to protect motoneurons from death induced by recombinant mouse IFNγ was first assessed in vitro in cultured motoneurons (FIG. 5). As described in Example 1, recombinant IFNγ, or sLIGHT, or agonistic anti-LT-βR antibodies, induced death of about 50% of motoneurons. Neutralizing goat polyclonal anti-IFNγ antibodies (15027, Sigma-Alrich) efficiently blocked IFNγ- or LIGHT-induced death of cultured motoneurons. IFNγ in combination with irrelevant rat IgG did not rescue motoneurons from death. Anti-IFNγ antibodies and irrelevant rat IgG alone were used as control.

Motoneurons were then co-cultured with wild-type and SOD1^{G93A} rat astrocytes. Inhibition of IFNγ activity with neutralizing anti-IFNγ antibodies (15027, Sigma-Alrich) did not significantly affect motoneuron survival in co-cultures of wild-type astrocytes but substantially prevented death of motoneurons induced by SOD1^{G93A} mutant astrocytes, to a higher extend than preventing LIGHT-LT-βR interaction with LT-βR-Fc decoy (Alexis Biochemicals) (FIG. 2).

Therefore, purified motoneurons are rescued from IFNγ-mediated neurotoxicity of astrocytes expressing ALS-linked mutant SOD1 by antagonizing the IFNγ-induced LIGHT-triggered death pathway. Those results support the beneficial use of IFNγ antagonists according to the invention for the treatment of selective death of motoneurons such as in ALS pathology.

### Cerebrospinal Fluid Injection of Function-Blocking Anti-IFNγ Antibody in Mice

90-day-old mice (early onset in B6SJL-TgN(SOD1-G93A)1Gur transgenic mice (G1H line)) (Gurney et al., 2004, Science 17, 1772-1775) were anesthetized and permanent 30-gauge stainless steel infusion catheter (Alzet Brain Infusion Kit 3; Durect Corp.) was stereotactically placed 0.3 mm anterior to bregma, 1 mm lateral and 2.6 mm below the surface of the skull. The catheter was secured to skull using glue and dental cement. The intra-cerebroventricular cannulae was connected to an Alzet osmotic pump (model 2004) through polyethylene tubing. The Alzet osmotic pump filled with 200-300 μg/ml of either rat monoclonal antagonistic anti-IFNγ antibody (clone R4-6A2) which specifically neutralizes mouse IFNγ (Havell, 1986, J. Interferon. Res., 6, 489-497) or with an irrelevant rat monoclonal antibody or the same isotype than the function-blocking antibody (IgG1). The alzet pump was implanted into a subcutaneous pocket in the midscapular area of the back of the mice. The solution of function-blocking or irrelevant control antibody is infused continuously at a rate of 0.25 μl/h for 4 weeks. Three weeks after implantation of a cannula infusing R4-6A2 IgG into the lateral ventricle of 13-week-old mice, a diffuse, intense immunoreactive staining was observed in widespread regions of brain, brainstem and spinal cord. IgG staining was observed in the striatum, cortex, external capsule, thalamus, and hippocampus, facial nuclei, cervical, thoracic and lumbar spinal cord. Neutralizing anti-IFNγ can be efficiently delivered to brain and spinal cord for up to 3 weeks.

### Behavioral Testing and Histopathological and Biochemical Analyses

The therapeutic benefit of the delivery of antagonistic anti-IFNγ antibody into the cerebrospinal fluid of SOD1-G93A mice through on disease progression and lifespan is determined by behavioural and histopathological analysis as previously described in Raoul et al., 2005, above, such as swimming tank and rotarod test, weight loss, counting surviving motoneuron in Cresyl violet stained spinal cords section, motor cortical abnormality, counting of motor fibers in osmium tetroxide-processed ventral roots. Weekly footprint analyses, performed weekly and expressed as distance in mm between forepaw and hindpaw, showed that anti-IFNγ immunotherapy significantly delayed the motor decline of SOD1^{G93A} mice. Irrelevant rat IgG were used as control. As expected, the therapeutic benefit of anti-IFNγ antibodies at early onset of disease was transient, the antibodies used in this experimental setting no longer being detected in the tissues four weeks post-implantation. The results support that IFNγ takes part in the pathogenic process and demonstrate the therapeutic potential of using IFNγ function-blocking molecules in motoneuron disease.

When motor decline is retarded, immunohistochemistry and immunoblotting in brain and spinal cord using appropriate markers and biochemical analysis are performed to confirm neuroprotection and further characterize which cellular and molecular events are associated with the protective effect. In particular, the extent of astrocyte and microglial activation is qualitatively and quantitatively examined in details by immunohistochemical methods (Raoul et al. 2005, above; Boillée et al., 2006, above). Quantitatively, upper motoneuron survival is studied on the basis of the degeneration of corticospinal tract (CST) in the dorsal column and dorsolateral CST of the spinal cord (Yamanaka et al., 2006, above).

### Intracerebroventricular Infusion of Function-Blocking Anti-IFNγ Antibody

A 30-gauge stainless steel infusion cannula (Alzet® Brain Infusion Kit 3; Durect Corp., CA, USA) was stereotaxically implanted in the lateral ventricle (0.3 mm anterior and 1 mm lateral relative to bregma; 2.5 mm below the surface of the skull) of 13 weeks old mice. The cannula was secured to skull using glue and dental cement and the Alzet osmotic pump (model 2004) was implanted into a subcutaneous pocket in the midscapular area of the back of the mice. Rat monoclonal antagonistic anti-IFNγ antibody (hybridoma product R4-6A2) or an irrelevant rat IgG₁ monoclonal antibody were diluted in PBS at a concentration of 300 μg/ml and infused intraventricularly at a continuous rate of 0.25 μl/hr.

### Foot Printing Analysis

Mice were tested weekly for footprint analysis. The forepaws and the hindpaws were inked with water-based non-toxic paints of two different colors. The mice were placed on a sheet of paper and allowed to walk through a tunnel. Footprint patterns were scanned and the distance between forepaw and hindpaw prints were calculated using the NIH ImageJ software. For each set of pawprints four measurements were taken.

### Example 4

### AAV Serotype 9 or 6-Mediated Delivery of IFNγ Inhibitors in a SOD1 Mutant ALS Mouse Model

The efficacy of a method and compositions according to the invention are tested through the therapeutic impact of motoneuron-specific IFNγ- and LIGHT-LT-βR-dependent death pathways inhibition, by blocking IFNγ or IFNγ effector LIGHT on motoneuron degeneration in a SOD1 mutant ALS mouse model via a non-replicative adeno-associated viral (AAV) or self-complementary AAV vector by intracerebral, intracerebrospinal, intramuscular, or systemic, injection. The therapeutic benefit of the AAV-mediated delivery of IFNγ/LIGHT inhibitors on disease progression and lifespan is determined by behavioural and histopathological analysis as described in Example 2.

### Viral Construct and Production

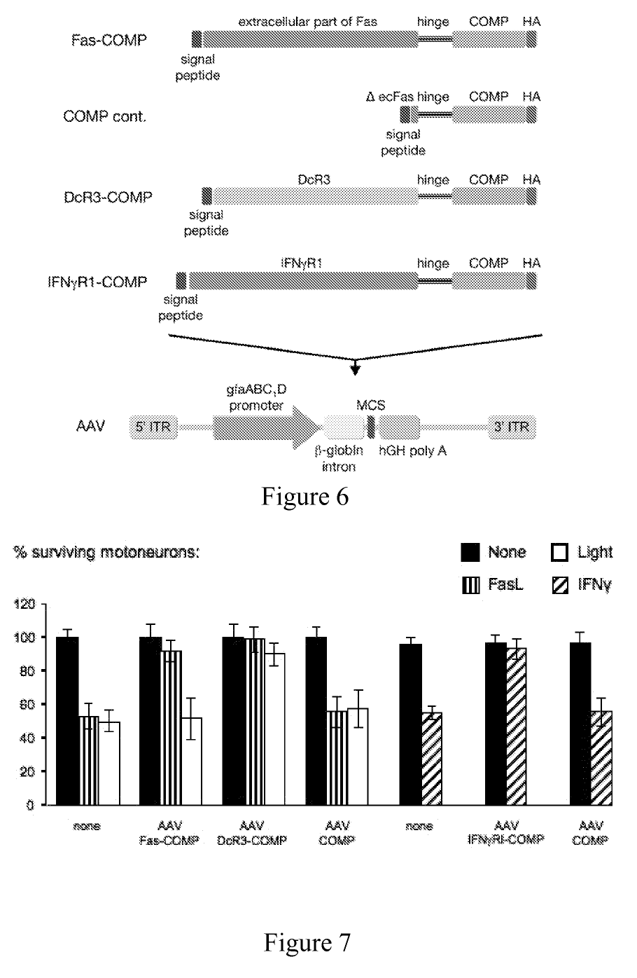

The following viral constructs were generated to drive expression in astrocytes of IFNγR1-COMP (nucleic acid of SEQ ID NO: 14, amino acid sequence of SEQ ID NO: 15), DcR3-COMP (nucleic acid of SEQ ID NO: 16, amino acid sequence of SEQ ID NO: 17), and a coiled-coil domain of cartilage oligomeric matrix protein (COMP) (nucleic acid of SEQ ID NO: 18, amino acid sequence of SEQ ID NO: 19) (negative control) under the control of the ubiquitously expressed phosphoglycerokinase (PGK1) (SEQ ID NO: 20) or GfaABC1-D (SEQ ID NO: 21), or cytomegalovirus (SEQ ID NO: 23), or chimeric CMV-chicken β-actin (SEQ ID NO: 22) promoters as described in Raoul et al., 2005, above (FIG. 6). IFNγR1-COMP was used for the inhibition of IFNγ/IFNγR interactions, and DcR3-COMP, which acts as a decoy for both FasL and LIGHT, was used for the inhibition of both the IFNγ-LIGHT-LT-βR and the Fas pathway. In addition, a Fas-COMP, composed of the extracellular part of the Fas fused to COMP, was also used for the inhibition of Fas/FasL, and a negative control was generated by deleting approximately 80% of the extra-cellular part of Fas (referred as secreted COMP, COMP). The above COMP which includes a linker peptide (PQPQPKPQPKPEPE of SEQ ID NO: 24), the pentamerisation domain of the cartilage oligomeric matrix protein (COMP) (Rattus norvegicus, Genbank accession number is NP_{—}036966 and Hemagglutinin sequence (HA tag sequence) at the C-terminal part (YPYDVPDYA of SEQ ID NO: 25), was used to develop dominant negative receptor approach based on this chimeric model, in order to efficiently inhibit LIGHT/LT-βR, and IFNγ-IFNγR interaction in vivo.

### Production of Functional AAV Vectors

Recombinant AAV vectors serotype 6 were produced by transient co-transfection of both the shuttle and pDF6 helper plasmid on 293AAV cell factories that stably expresses the E1 gene needed for activation of rep and cap promoters (Grimm et al., 2003, Mol. Ther., 7, 839-850) according to standard procedures, and concentrated virus suspensions obtained by liquid chromatography (LC) on heparin affinity columns (Towne et al., 2008, above).

To produce AAV9 vectors, the cap-6 sequence coding for serotype 6 VP1, VP2 and VP3, was replaced by the cap-9 sequence to generate the pDF9 helper plasmid (Cearley et al., 2008, Mol. Ther., 19, 1359-1368; Bish et al., 2008, Mol. Ther., 16, 1953-1959). The functionality of AAV9 production was tested by monitoring expression from a fluorescent EGFP expression cassette on 293T and HeLa cell lines by flow cytometry. The efficacy of packaging was measured by real-time PCR on virus suspension as described (Towne et al. 2008, above).

The constructs were checked by sequencing. The efficacy of IFNγR1-COMP, DcR3-COMP, Fas-COMP, and COMP to be efficiently secreted by astrocytes and to specifically interact with their respective ligands were evaluated in vitro by immunoprecipitation assays. Glioma cell lines were transduced with vectors encoding IFNγR1-COMP, DcR3-COMP, Fas-COMP, or COMP and conditioned media collected. Recombinant soluble LIGHT and IFNγ were added to different conditioned media. Immunoprecipitation of ligand-receptor complex were performed using anti-hemagglutinin (HA) tag antibody, since these constructs include an HA tag at their C-terminus, and immunocomplexes analysed by SDS-PAGE followed by immunoblotting with antibodies to LIGHT, IFNγR1 and IFNγ. Immunoprecipitation results demonstrated that the IFNγR1-, DcR3- and Fas-COMP chimeric receptors effectively interact with their corresponding recombinant ligand, consistent with a dominant negative effect.

IFNγR1-COMP or DcR3-COMP Rescue Motoneurons from Light/IFNγ-Induced Death

The functionality of the dominant negatives in inhibiting Fas and LIGHT/IFNγ-induced death was evaluated in an isolated motoneurons culture system as described in Example 2. Embryonic motoneurons isolated from spinal cords of E12.5 CD1 mice were cultured for 6 h and then infected with 50 TU/ml of indicated with AAV-IFNγR1-COMP, AAV-DcR3-COMP, AAV-Fas-COMP, or AAV-COMP viral vectors. After 6 h of incubation the culture medium was replaced and motoneurons were treated 48 h later (when indicated) with effective doses of either sFasL (50 ng/ml), sLight (50 ng/ml) or IFNγ (250 ng/ml). Forty-eight hours later, survival was determined by direct counting of motoneurons (FIG. 7). As expected, Fas-COMP efficiently rescued motoneurons from sFasL-induced death but did not confer any protective effect on motoneurons against LIGHT killing effect while DcR3-COMP rescued motoneurons from both sFasL- and sLIGHT-induced death. On the other hand, IFNγ-triggered death was blocked by IFNγR1-COMP. The negative control COMP (on FIG. 7) did not interfere with death induced by either ligands or cytokine

### Animals and Viral Vector Administration

AAV6 encoding for IFNγR1-COMP and COMP control using the PGK1 promoter described above are delivered by intramuscular, intracerebrospinal, or systemic injection into SOD1 mutant mice. Mice are injected (2.5 10⁵-10⁶ TU) in hindlimb, forelimb, dorsal and facial muscles to ensure an optimal delivery of therapeutic information along the spinal cord and brainstem. We observed that AAV6 intramuscular injections led to the transduction of approximately 30% of motoneurons (Duplan et al., 2010, J. Neurosci. 30, 785-796) and systemic AAV6 injections to about 5% motoneuron transduced (Towne et al., 2008, Mol. Ther. 16, 1018-1025). AAV9 encoding IFNγR1-COMP, DcR3-COMP, Fas-COMP, or COMP control using a CMV promoter or the GfaABC1-D promoter are delivered by intravascular (in the tail vein) and intramuscular injection, at a starting dose of 4×10¹² genome copies. Systemic AAV9 adminsitration has been observed to lead to the transduction of 70% of motoneurons (Wang et al., 2010, Mol. Ther., 28 Sep. 2010) Administration of AAVs are performed before the appearance of motor impairment (non-symptomatic stage, 40 days) and at the time of disease onset (90 days), a more stringent situation, but which is closed to the clinical reality.

The pattern of infection obtained following systemic and intramuscular injection of AAV6 or 9 vectors coding for a reporter EGFP-expressing cassette is monitored by immunohistology as described in Example 2 and allows to determine which cell types express the transgene, particularly at the level of the spinal cord and motor cortex, using the above specific markers for motoneurons (VAChT) and astroglial/microglial cells (GFAP, Iba1). Concerning cortical motoneurons, non-phosphorylated neurofilament (SMI32) staining, their location in the layer V of the cortex and their size will be used for their identification. Liver, heart, spleen and skeletal muscles, will also be studied for their expression of EGFP.

### Example 5

### Disease Progression is Delayed and Lifespan Expanded in LIGHT-Deficient SOD1^{G93A} Mice

The therapeutic impact of the inhibition of motoneuron-specific IFNγ-induced LIGHT-LT-βR-dependent death pathways is evaluated by deleting LIGHT in a SOD1 mutant ALS mouse model. The therapeutic benefit of LIGHT ablation on disease progression and lifespan is determined by behavioural and histopathological analysis as described in Example 2.

### Genetic Ablation of LIGHT in SOD1^{G93A} Mice Retards Disease Progression

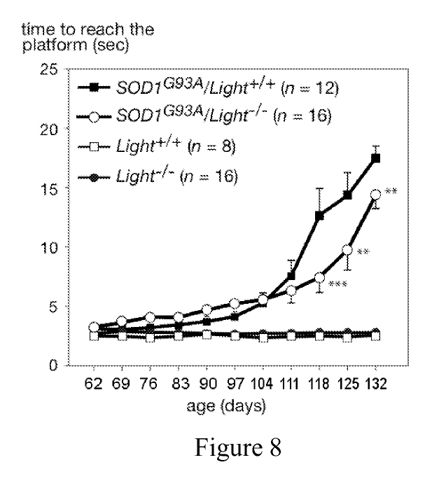

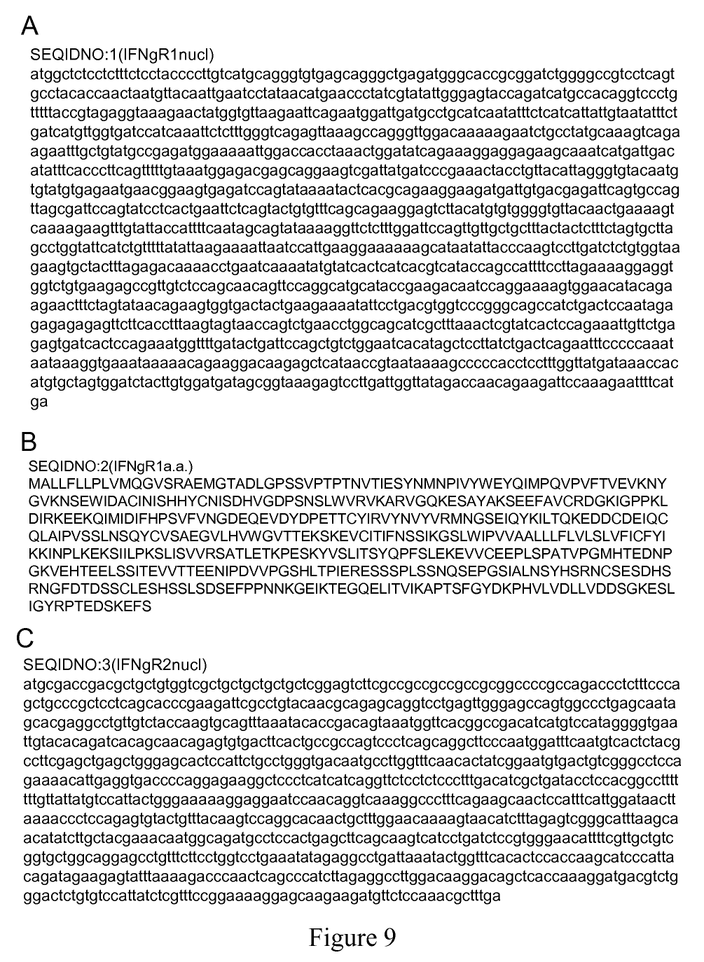

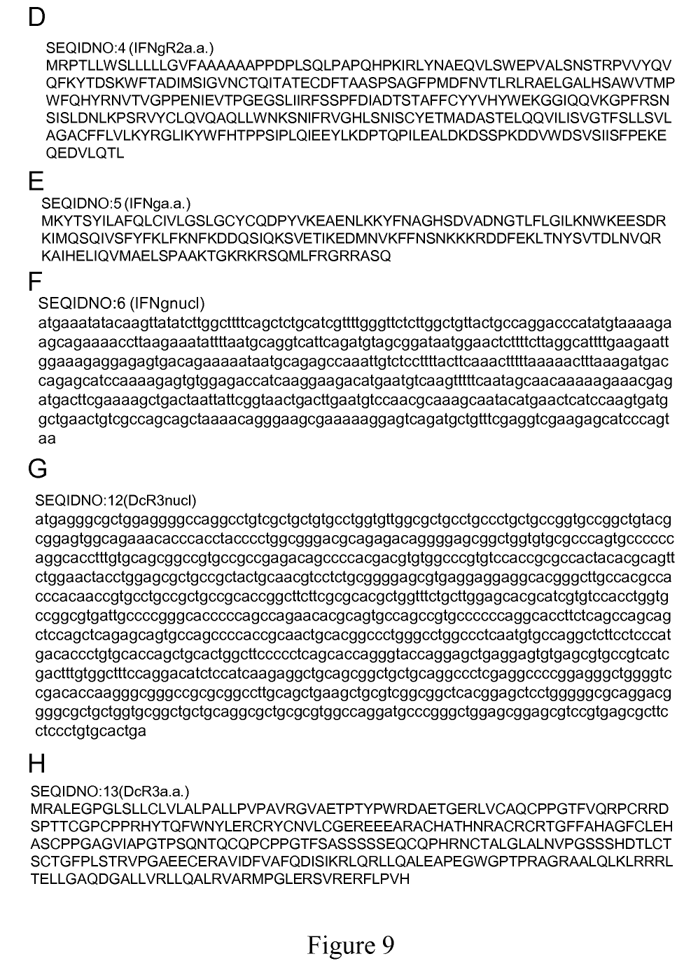

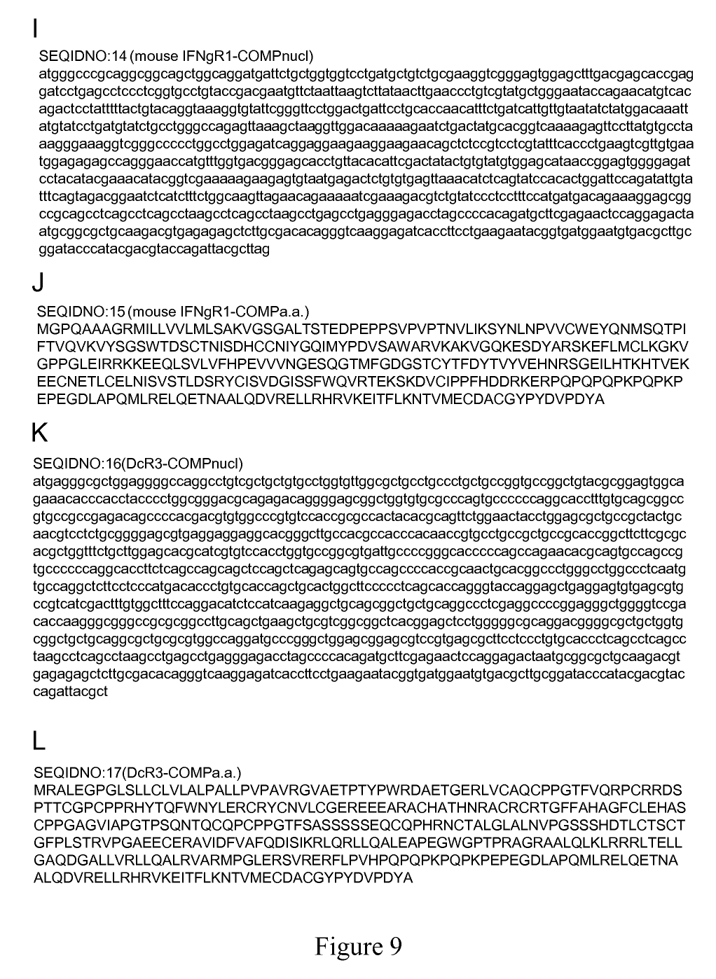

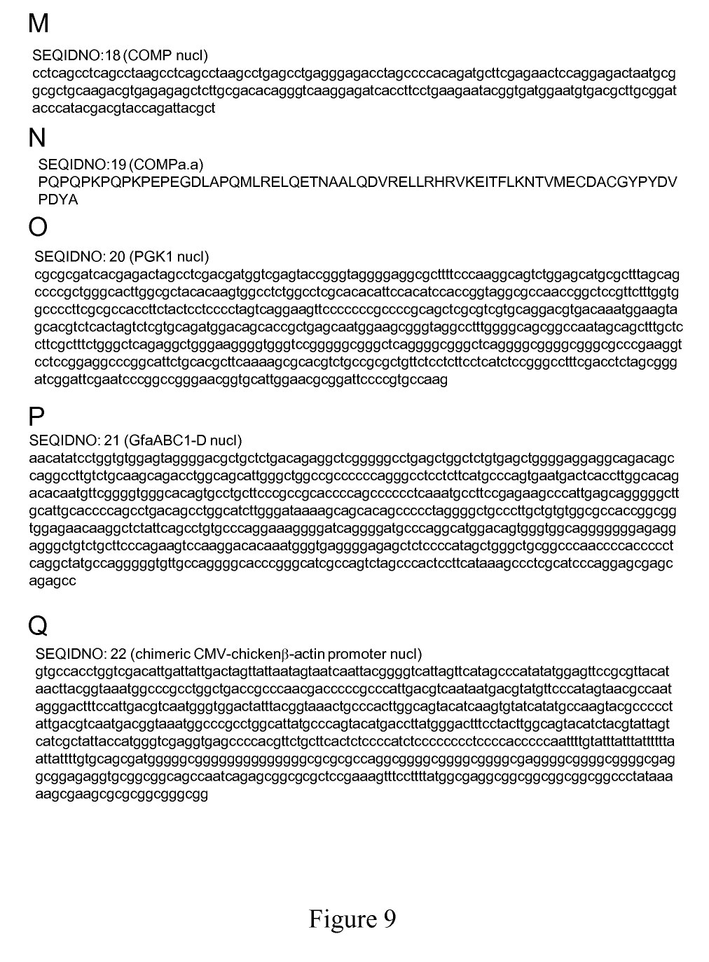

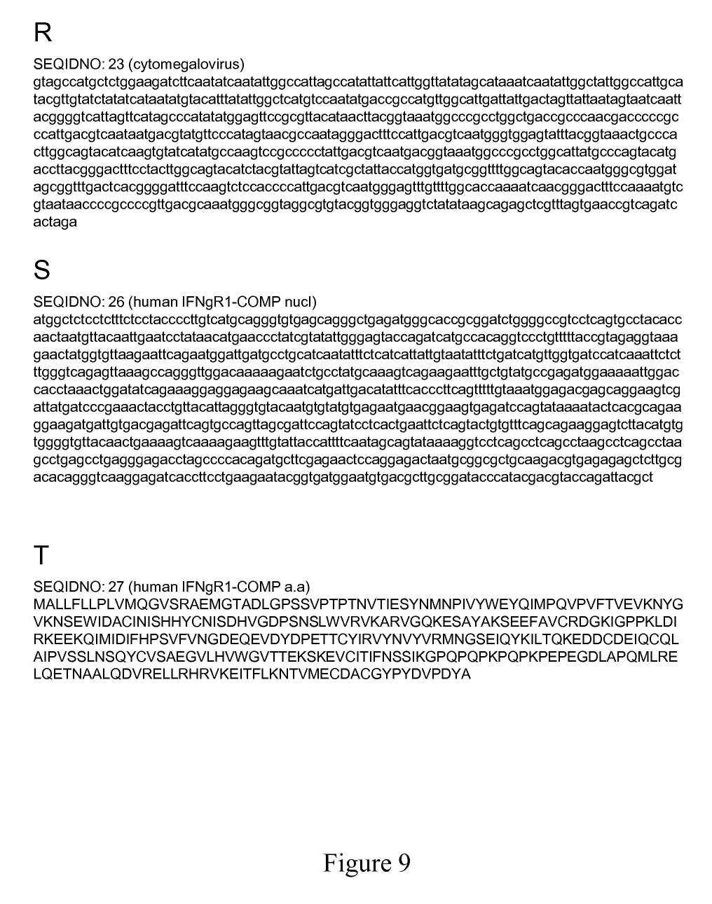

To evaluate the functional involvement of the IFNγ-LIGHT-LT-βR pathway in ALS pathogenesis, LIGHT was genetically deleted in mice overexpressing SOD1^{G93A} by cross-breeding (SOD1^{G93A}/LIGHT−/−). LIGHT deficient mice are viable and fertile with no behavioral abnormalities (Scheu et al., 2002, J. Exp. Med., 195, 1613-1624). As described in Boillee et al., 2006, Science, 312, 1389-1392), the cumulative probability of onset of SOD1^{G93A}/LIGHT+/+, SOD1^{G93A}/LIGHT+/−, and SOD1^{G93A}/LIGHT−/− mice, was assessed via the peak of weight curve. No significant difference between SOD1^{G93A}/LIGHT+/+ and SOD1^{G93A}/LIGHT+/− mice was observed. Disease progression was then evaluated weekly in the cohorts of LIGHT+/+, LIGHT−/−, SOD1^{G93A}/LIGHT+/+, and SOD1^{G93A}/LIGHT−/− mice, by measuring the swimming performance of mice, which is dependent on the frequency and strength of hind limb kicking (Raoul et al., 2005, Nat. Med., 11, 423-428). LIGHT deficiency significantly retarded the decline of motor function in ALS mice (FIG. 8A). Kaplan-Meier survival curves for SOD1^{G93A}/LIGHT+/+ and SOD1^{G93A}/LIGHT−/− mice also showed that LIGHT deletion increased the lifespan of SOD1^{G93A} mutant mice by 17.9 days.

### Genetic Ablation of LIGHT in SOD1^{G93A} Mice Protects Against Motoneurons Death

The potential association of the amelioration in motor performance of LIGHT deficient SOD1 mice with an increase in motoneuron survival was investigated. The number of surviving motoneurons was quantified on VAChT-immunostained sections as described in Example 2 taken from the lumbar region of 120-days-old mice spinal cords of different genotypes. While, at this stage, a marked loss of lumbar motoneurons can be seen in SOD1^{G93A}/LIGHT+/+ mice, a significant increase in the number of surviving motoneurons in SOD1^{G93A}/LIGHT−/− mice was observed.

Analysis of LIGHT and SOD1^{G914} Mutant Mice LIGHT^{-/-} and SOD1^{G93A} mice were genotyped as previously described (Duplan et al., 2010, J. Neurosci., 30, 785-796; Scheu et al., 2002, above). LIGHT−/− male mice were crossed with SOD1^{G93A} female mice to obtain SOD1^{G93A}/LIGHT+/− mice. SOD1^{G93A}/LIGHT+/− male mice were then backcrossed with LIGHT+/− female mice. Following the double cross-breeding, only LIGHT+/+, LIGHT−/−, SOD1^{G93A}/LIGHT+/+ and SOD1^{G93A}/LIGHT−/− mice were chosen for the behavioral assays. To define the onset of disease, the body weight was measured every 2 days from day 50 and determined when the weight curve reached a plateau (Yamanaka et al., 2008, Nat. Neurosci., 11, 251-253). To assess progression of motor decline, a swimming tank test was performed starting at the age of 50 days and measured swimming speed as previously described (Raoul et al., 2005, above). For statistical purposes, the maximum swimming latency was set at 20 seconds (s). The mortality was defined as the point in time when the mice are unable to right themselves within 30 s after being placed upon their back. All behavioral studies were done in a blinded manner.

### Statistical Analysis

Statistical significance was determined as described in Example 2. Statistical analysis of swimming performance was done using a two-way (group×time) repeated measures ANOVA followed by a Newman-Keuls's post hoc test. A log-rank test was used to calculate the statistical differences in the onset and survival of the different mouse cohorts. Kaplan-Meier survival curves were plotted using GraphPad Prism™ Software (GraphPad Software, Inc., USA). GraphPad Prism™ and StatSoft™ Statistica software (StatSoft, Inc., USA) were used for calculations. Significance was accepted at the level of P<0.05.

Altogether, results indicate that LIGHT contributes to disease progression, but not disease onset, in ALS mice, and that LIGHT deletion protects against motoneurons degeneration and significantly retards progressive motor deficit and death in SOD1 mice, underlining the functional involvement of the IFNγ-LIGHT-LT-βR pathway in ALS pathogenesis.

1. (canceled)

2. The method of claim 29, wherein the INFγ antagonist is selected from an INFγ antibody, an INFγ antibody fragment, an INFγ aptamer, an INFγ chimeric protein and a viral vector.

3. The method of claim 29, wherein the INFγ antagonist is an INFγ antibody.

4. The method of claim 29, wherein the INFγ antagonist is a viral vector.

5. A method of treating a motoneuron disease or disorder in a subject in need thereof, comprising administering in said subject a pharmaceutical composition which comprises an INFγ antagonist.

6. A method according to claim 5 wherein the INFγ antagonist is selected from an INFγ antibody, an INFγ aptamer, an INFγ chimeric protein and a viral vector.

7. A method according to claim 6 wherein the INFγ antagonist is an INFγ antibody.

8. A method according to claim 6 wherein the INFγ antagonist is viral vector.

9. A method for delivering a nucleic acid sequence encoding an INFγ antagonist to cells selected from neural, microglial and meningeal cells comprising:
(a) Providing a virion comprising a viral vector, said vector comprising at least one expression control element operably linked to a nucleic acid sequence encoding for an INFγ antagonist;
(b) Bringing the virion into contact with the said cells, whereby transduction of the viral vector results in the expression of said nucleic acid sequence in the transduced cells and the expression of said nucleic acid sequence by said cells.

10. A viral vector comprising at least one expression control element operably linked to a nucleic acid sequence encoding for an INFγ antagonist, wherein the nucleic acid sequence encoding for an INFγ antagonist comprises a nucleic acid sequence encoding for INFγRI (SEQ ID NO: 2) and a nucleic acid sequence encoding for an oligomerisation domain (SEQ ID NO: 19) or a variant thereof being at least 80% identical to SEQ ID NO: 19.

11. A viral vector according to claim 10 wherein the nucleic acid sequence encoding for an INFγ antagonist encodes for INFγ RI-COMP (SEQ ID NO: 27) or a variant thereof being at least 80% identical to SEQ ID NO: 27.

12. A viral vector according to claim 10 wherein the nucleic acid sequence encoding for an INFγ antagonist encodes for INFγRI-COMP and has a sequence consisting of SEQ ID NO: 26.

13. A viral vector comprising at least one expression control element operably linked to a nucleic acid sequence encoding for an INFγ antagonist, wherein the nucleic acid sequence encoding for an INFγ antagonist comprises a nucleic acid sequence encoding for DcR3 (SEQ ID NO: 13) and a nucleic acid sequence encoding for an is oligomerisation domain (SEQ ID NO: 19) or a variant thereof being at least 80% identical to SEQ ID NO: 19.

14. A viral vector according to claim 13 wherein the nucleic acid sequence encoding for an INFγ antagonist encodes for DcR3-COMP (SEQ ID NO: 17) or a variant thereof being at least 80% identical to SEQ ID NO: 17.

15. A viral vector according to claim 13 wherein the nucleic acid sequence encoding for an INFγ antagonist encodes for DcR3-COMP and has a sequence consisting of SEQ ID NO: 16.

16. A viral vector according to claim 10, wherein the viral vector is a rAAV comprising capsid proteins of AAV serotype 9 or 6.

17. A viral vector according to claim 10, wherein the expression control element is a ubiquitous, a neuronal- or a glial-specific promoter.

18. A viral vector according to claim 10, wherein the expression control element is a ubiquitous, a neuronal- or a glial-specific promoter selected from phosphoglycerokinase (PGK1) (SEQ ID NO: 20) or GfaABCI-D (SEQ ID NO: 21), or cytomegalovirus (SEQ ID NO: 23), or chimeric CMV-chicken β-actin (SEQ ID NO: 22).

19. A viral vector according to claim 10 for use as a medicament.

20. A viral vector according to claim 10 for the treatment of a motoneuron disease or disorder.

21. A pharmaceutical preparation comprising at least one viral vector according to claim 10 and pharmaceutically acceptable carrier or excipient.

22. (canceled)

23. The method of claim 29, wherein the motoneuron disease or disorder is amyotrophic lateral sclerosis.

24. (canceled)

25. An in vitro method for detection and/or prognosis of a motoneuron disease in a sample from a subject, comprising the following steps: (a) measuring INFγ levels in a sample from said subject; and (b) comparing INFγ level data obtained in step (a) to INFγ level data of subjects suffering from a motoneuron disease, wherein INFγ levels correlate with a motoneuron disease status in said subject.

26. A method according to claim 25 wherein the sample is selected from a serum sample, a cerebrospinal fluid sample or a tear sample.

27. A method according to claim 9, wherein expression of said nucleic acid sequence occurs in the transduced cells and the expression of said nucleic acid sequence by cells result in the reduction of motoneuron damage.

28. A method according to claim 5 wherein the INFγ antagonist is a viral vector comprising at least one expression control element operably linked to a nucleic acid sequence encoding for an INFγ antagonist, wherein the nucleic acid sequence encoding for an INFγ antagonist comprises a nucleic acid sequence encoding for INFγRI (SEQ ID NO: 2) and a nucleic acid sequence encoding for an oligomerisation domain (SEQ ID NO: 19) or a variant thereof being at least 80% identical to SEQ ID NO: 19.

29. A method of treating a motoneuron disease or disorder in a subject in need thereof, comprising administering in said subject a pharmaceutical composition which comprises (a) a pharmaceutically acceptable excipient; and (b) virions comprising a viral vector, said viral vector comprising a nucleic acid sequence encoding for an INFγ antagonist, operably linked to at least one expression control element that controls expression of the said nucleic acid sequence in an amount effective to treat the motoneuron disease or disorder in said subject.
